(19) United States
(12) Patent Application Publication
(10) Pub. No.: US 2004/0091850 A1
(43) Pub. Date: May 13, 2004

(54) Single cell analysis of membrane molecules

(76) Inventors: Travis Boone, Los Altos, CA (US); Sharat Singh, San Jose, CA (US)

Correspondence Address: ACLARA BIOSCIENCES, INC., 1288 PEAR AVENUE, MOUNTAIN VIEW, CA, 94043, US

(21) Appl. No.: 10/702,879

(22) Filed: Nov. 5, 2003

Related U.S. Application Data
(60) Provisional application No. 60/425,129, filed on Nov. 8, 2002.

(51) Int. Cl. C12Q 1/00; G01N 33/53; G01N 33/567

(52) U.S. Cl. 435/4; 435/7.2

(57) ABSTRACT

Interrogation of surface receptors of individual cells within a population of cell types in a single mixture is described. Each cell comprises, bound to target surface receptors on the cell, a collection of binding compositions, each comprising a distinct molecular tag. A continuous flow of cell medium containing the cells is pneumatically transported through a channel in a separation device, toward a cleavage/release zone, where specific tags correlating to each binding composition, and thus to each corresponding cell surface receptor, are released from the binding compositions on the cell surface. The method is effective to produce a separately detectable collection of tag signals for each cell.

This application claims priority from U.S. provisional application Ser. No. 60/425,129 filed 8 Nov. 2002.

### FIELD OF THE INVENTION

The present invention relates generally to methods and systems for detecting molecules on surfaces of single cells, and more particularly, to methods and systems using binding compositions having releasable molecular tags for analyzing surface molecules of single cells in nanoliter and subnanoliter volumes.

### BACKGROUND OF THE INVENTION

The interactions of cell surface membrane components play crucial roles in transmitting extracellular signals to a cell in normal physiology, and in disease conditions. In particular, different expression levels or different dimerization or oligomerization states of cell surface receptors often appear to be associated with several disease conditions, e.g. George et al, Nature Reviews Drug Discovery, 1: 808-820 (2002); Mellado et al, Ann. Rev. Immunol., 19: 397-421 (2001); Schlessinger, Cell, 103: 211-225 (2000); Yarden, Eur. J. Cancer, 37: S3-S8 (2001). The role of such cell surface events in diseases, such as cancer, has been the object of intense research and has led to the development of several new drugs and drug candidates, e.g. Herbst and Shin, Cancer, 94: 1593-1611 (2002); Yarden and Sliwkowski, Nature Reviews Molecular Cell Biology, 2: 127-137 (2001).

A wide variety of techniques have been used to study cell surface membrane components, including immunoprecipitation, chemical cross-linking, yeast two-hybrid systems, tagged fusion proteins, bioluminescence resonance energy transfer (BRET), fluorescence resonance energy transfer (FRET), mass spectroscopy, and the like, e.g. Golemis, editor, Protein-Protein Interactions (Cold Spring Harbor Laboratory Press, New York, 2002); Price et al (cited above); Sorkin et al, Curr. Biol., 10: 1395-1398 (2000); McVey et al, J. Biol. Chem., 17: 14092-14099 (2001); Salim et al, J. Biol. Chem., 277: 15482-15485 (2002); Angers et al, Proc. Natl. Acad. Sci., 97: 3684-3689 (2000); Jones et al, Proteomics, 2: 76-84 (2002); and Petricoin III, et al, The Lancet, 359: 572-577 (2002). Unfortunately, such techniques are frequently difficult to apply, generally lack sufficient sensitivity to provide an accurate profile of component populations, such as receptors, and cannot measure multiple surface components or interacting surface components, which are crucial for the activation of cellular processes. Presently available techniques are also limited because analyses typically take place on pools of cells so that cell-to-cell differences are lost as the heterogeneity of individual cell characteristics is averaged across a population, e.g. Sims et al, Current Opinion in Biotechnology, 14: 23-28 (2003). Although there are several flow cytometric technologies that do provide measurements on single cells, current instruments lack sensitivity or cannot measure interaction of surface components, such as receptor dimerization, Fu et al, Nature Biotechnology, 17: 1109-1111 (1999); Li et al, Anal. Chem. 69: 1564-1568 (1997); Perez et al, Nature Biotechnology, 20: 155-162 (2002); Szollosi et al, J. Biotechnol., 82: 251-266 (2002); McCain et al, Anal. Chem., 75: 5646-5655 (2003).

In view of the above, the availability of an convenient and sensitive assay for measuring cell surface membrane molecules on single cells would be highly desirable.

### SUMMARY OF THE INVENTION

The invention is directed to methods and systems for interrogating membrane-associated analytes, e.g. surface membrane receptors, of individual cells in a sample. In accordance with the method, binding compounds having releasable molecular tags are contacted with a cell mixture, such that the binding compounds bind to targeted membrane-associated analytes in the cell mixture. Cell medium containing such cells is then caused to flow so that cells are transported through a channel in a microfluidics device to a cleavage zone, which is adjacent to a separation channel. At the cleavage zone, molecular tags are released from the binding compounds and are electrophoretically separated in the separation channel. Therefore, for every cell that passes through the cleavage zone, a cluster of released tags is electrophoretically separated and transported toward a detection point. At the detection point, signals from release molecular tags from each individual cell are sensed and quantified in a continuous fashion.

The method allows rapid, multiplexed cell-by-cell analysis with high sensitivity. Applications of the method are varied and include, for example, cancer research and testing of host cell responses for defense against biological pathogens.

### BRIEF DESCRIPTION OF THE DRAWINGS

FIGS. 1A-1G illustrate microfluidic systems for implementing the method of the invention.

FIGS. 2A-2B illustrate diagrammatically methods for attaching molecular tags to antibodies.

FIGS. 3A-3F illustrate oxidation-labile linkages and their respective cleavage reactions mediated by singlet oxygen.

FIGS. 4A-4J show the structures of tags that have been designed and synthesized.

FIGS. 5A-5D illustrate the chemistries of synthesis of the tag moieties illustrated in FIG. 4.

### DEFINITIONS

“Antibody” means an immunoglobulin that specifically binds to, and is thereby defined as complementary with, a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, Fab′, and the like. In addition, aggregates, polymers, and conjugates of immunoglobulins or their fragments can be used where appropriate so long as binding affinity for a particular polypeptide is maintained.

“Antibody binding composition” means a molecule or a complex of molecules that comprises one or more antibodies, or fragments thereof, and derives its binding specificity from such antibody or antibody fragment. Antibody binding compositions include, but are not limited to, (i) antibody pairs in which a first antibody binds specifically to a target molecule and a second antibody binds specifically to a constant region of the first antibody; a biotinylated antibody that binds specifically to a target molecule and a streptavidin protein, which protein is derivatized with moieties such as molecular tags or photosensitizers, or the like, via a biotin moiety; (ii) antibodies specific for a target molecule and conjugated to a polymer, such as dextran, which, in turn, is derivatized with moieties such as molecular tags or photosensitizers, either directly by covalent bonds or indirectly via streptavidin-biotin linkages; (iii) antibodies specific for a target molecule and conjugated to a bead, or microbead, or other solid phase support, which, in turn, is derivatized either directly or indirectly with moieties such as molecular tags or photosensitizers, or polymers containing the latter.

“Antigenic determinant,” or “epitope” means a site on the surface of a molecule, usually a protein, to which a single antibody molecule binds; generally a protein has several or many different antigenic determinants and reacts with antibodies of many different specificities. A preferred antigenic determinant is a phosphorylation site of a protein.

“Binding moiety” means any molecule to which molecular tags can be directly or indirectly attached that is capable of specifically binding to an analyte. Binding moieties include, but are not limited to, antibodies, antibody binding compositions, peptides, proteins, nucleic acids, and organic molecules having a molecular weight of up to 1000 daltons and consisting of atoms selected from the group consisting of hydrogen, carbon, oxygen, nitrogen, sulfur, and phosphorus. Preferably, binding moieties are antibodies or antibody binding compositions.

“Capillary-sized” in reference to a separation column means a capillary tube or channel in a plate or microfluidics device, where the diameter or largest dimension of the separation column is between about 25-500 microns, allowing efficient heat dissipation throughout the separation medium, with consequently low thermal convection within the medium.

“Complex” as used herein means an assemblage or aggregate of molecules in direct or indirect contact with one another. As used herein, “contact,” or more particularly, “direct contact” in reference to a complex of molecules, or in reference to specificity or specific binding, means two molecules are close enough that weak noncovalent chemical interactions, such as Van der Waal forces, hydrogen bonding, ionic and hydrophobic interactions, and the like, dominate the interaction of the molecules. Generally, a complex of molecules is stable in that under assay conditions the complex is thermodynamically more favorable than a non-aggregated state of its component molecules.

“ErbB receptor” means a human receptor protein of the set including Her1 (also referred to as epidermal growth factor receptor, or EGFR, or ErbB1), Her2 (also referred to as ErbB2), Her3 (also referred to as ErbB3), Her4 (also referred to as ErbB4), and proteins having substantially identical amino acid sequences thereof. Her1, Her2, Her3, and Her4 are described under NCBI accession numbers NP_{—}005219; NP_{—}004439 or P04626; NP_{—}001973; and NP_{—}005226; respectively.

The term “isolated” in reference to a polypeptide or protein means substantially separated from the components of its natural environment. Preferably, an isolated polypeptide or protein is a composition that consists of at least eighty percent of the polypeptide or protein identified by sequence on a weight basis as compared to components of its natural environment; more preferably, such composition consists of at least ninety-five percent of the polypeptide or protein identified by sequence on a weight basis as compared to components of its natural environment; and still more preferably, such composition consists of at least ninety-nine percent of the polypeptide or protein identified by sequence on a weight basis as compared to components of its natural environnent. Most preferably, an isolated polypeptide or protein is a homogeneous composition that can be resolved as a single spot after conventional separation by two-dimensional gel electrophoresis based on molecular weight and isoelectric point. Protocols for such analysis by conventional two-dimensional gel electrophoresis are well known to one of ordinary skill in the art, e.g. Hames and Rickwood, Editors, Gel Electrophoresis of Proteins: A Practical Approach (RL Press, Oxford, 1981); Scopes, Protein Purification (Springer-Verlag, New York, 1982); Rabilloud, Editor, Proteome Research: Two-Dimensional Gel Electrophoresis and Identification Methods (Springer-Verlag, Berlin, 2000).

“Membrane-associated analyte” means a substance, compound, molecule, or component or part of any of the foregoing that is directly or indirectly attached to a membrane, especially a biological membrane such as the cell surface membrane of a mammalian cell or tissue. The attachment may be direct, for example, when a membrane-associated analyte has a lipophilic moiety, or is attached to another molecule that has a lipophilic moiety, capable of anchoring it in a membrane. The attachment may also be indirect, for example, when a membrane-associated analyte is a soluble ligand that binds to, and forms a stable complex with, a cell surface receptor. A membrane-associated analyte may be, but is not limited to, a peptide, protein, polynucleotide, polypeptide, oligonucleotide, organic molecule, hapten, epitope, part of a biological cell, a posttranslational modification of a protein, a receptor, a complex sugar attached to a membrane component such as a receptor, a soluble compound forming a stable complex with a membrane such as a vitamin, a hormone, a cytokine, or the like, forming and the like. There may be more than one analyte associated with a single molecular entity, e.g. different phosphorylation sites on the same protein. Membrane-associated analytes include cell surface molecules, such as cell membrane receptors. In one aspect of the invention, membrane-associated analytes are cell membrane receptors selected from the group consisting of receptor tyrosine kinases and Gprotein coupled receptors. In another aspect, membrane-associated analytes include receptor tyrosine kinases, such as Her receptor dimers, IGF-1R, VEGFR1(Flt1)-VEGFR2(KDR), VEGFR2(KDR)-VEGFR2(KDR), PDGFRα-PDGFRα, PDGFRα-PDGFRβ, PDGFRβ-PDGFRβ, Kit/SCFR homodimers, an FGFR dimer, NGFR(TrkA)-NGFR(TrkA), α₂-adrenergic receptor homodimer, α₂-adrenergic-β₂-adrenergic receptor heterodimer, β₂-adrenergic receptor homodimer, GABA_{B}R1-GABA_{B}R2 receptor heterodimer, ATII receptor homodimer, cholescystokinin-dopamine receptor heterodimer, bradykininin B2 receptor homodimer, M2-M3 muscarinic receptor heterodimer, CCR2 receptor homodimer, μ-β opioid receptor heterodimer, D1 dopamine receptor homodimer, 5-HT 1B-5-HT 1D receptor heterodimer, D2 dopamine receptor homodimer, α_{2c}-adrenergic-M3 muscarinic receptor heterodimer, D3 dopamine receptor homodimer, and β₂-adrenergic-δ opioid receptor heterodimer. In one aspect of the invention, Her dimers are selected from the group consisting of dimers is selected from the group consisting of Her1-Her1, Her1-Her2, Her1-Her3, Her1-Her4, Her2-Her2, Her2-Her3, Her2-Her4, Her3-Her4, and Her4-Her4 dimers. “Dimer” in reference to membrane-associated analytes means a stable complex of two membrane-associated analytes. A dimer of membrane-associated analytes may form as the result of interaction with a ligand, i.e. ligand-induced dimerization, e.g. Schlessinger, Cell, 110: 669-672 (2002). “Oligomer” or “cluster” in reference to membrane-associated analytes means a stable, usually non-covalent, association of at least two membrane-associated analytes.

“Polypeptide” refers to a class of compounds composed of amino acid residues chemically bonded together by amide linkages with elimination of water between the carboxy group of one amino acid and the amino group of another amino acid. A polypeptide is a polymer of amino acid residues, which may contain a large number of such residues. Peptides are similar to polypeptides, except that, generally, they are comprised of a lesser number of amino acids. Peptides are sometimes referred to as oligopeptides. There is no clear-cut distinction between polypeptides and peptides. For convenience, in this disclosure and claims, the term “polypeptide” will be used to refer generally to peptides and polypeptides. The amino acid residues may be natural or synthetic.

“Protein” refers to a polypeptide, usually synthesized by a biological cell, folded into a defined three-dimensional structure. Proteins are generally from about 5,000 to about 5,000,000 or more in molecular weight, more usually from about 5,000 to about 1,000,000 molecular weight, and may include posttranslational modifications, such acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, farnesylation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, phosphorylation, prenylation, racemization, selenoylation, sulfation, and ubiquitination, e.g. Wold, F., Post-translational Protein Modifications: Perspectives and Prospects, pgs. 1-12 in Post-translational Covalent Modification of Proteins, B. C. Johnson, Ed., Academic Press, New York, 1983. Proteins include, by way of illustration and not limitation, cytokines or interleukins, enzymes such as, e.g., kinases, proteases, galactosidases and so forth, protamines, histones, albumins, immunoglobulins, scleroproteins, phosphoproteins, mucoproteins, chromoproteins, lipoproteins, nucleoproteins, glycoproteins, T-cell receptors, proteoglycans, and the like.

The term “sample” means a quantity of material that is suspected of containing one or more molecular complexes that are to be detected or measured. As used herein, the term includes a specimen (e.g., a biopsy or medical specimen, also referred to as a “patient sample”) or a culture (e.g., microbiological culture). It also includes both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention. In particular, biological samples include fixed biological specimens, such as patient biopsy specimens treated with a fixative, biological specimens embedded in paraffin, frozen biological specimens, smears, and the like.

A “separation profile” in reference to the separation of molecular tags means a chart, graph, curve, bar graph, or other representation of signal intensity data versus a parameter related to the molecular tags, such as retention time, mass, or the like, that provides a readout, or measure, of the number of molecular tags of each type produced in an assay. A separation profile may be an electropherogram, a chromatogram, an electrochromatogram, a mass spectrogram, or like graphical representation of data depending on the separation technique employed. A “peak” or a “band” or a “zone” in reference to a separation profile means a region where a separated compound is concentrated. There may be multiple separation profiles for a single assay if, for example, different molecular tags have different fluorescent labels having distinct emission spectra and data is collected and recorded at multiple wavelengths. In one aspect, released molecular tags are separated by differences in electrophoretic mobility to form an electropherogram wherein different molecular tags correspond to distinct peaks on the electropherogram. A measure of the distinctness, or lack of overlap, of adjacent peaks in an electropherogram is “electrophoretic resolution,” which may be taken as the distance between adjacent peak maximums divided by four times the larger of the two standard deviations of the peaks. Preferably, adjacent peaks have a resolution of at least 1.0, and more preferably, at least 1.5, and most preferably, at least 2.0. In a given separation and detection system, the desired resolution may be obtained by selecting a plurality of molecular tags whose members have electrophoretic mobilities that differ by at least a peak-resolving amount, such quantity depending on several factors well known to those of ordinary skill, including signal detection system, nature of the fluorescent moieties, the diffusion coefficients of the tags, the presence or absence of sieving matrices, nature of the electrophoretic apparatus, e.g. presence or absence of channels, length of separation channels, and the like. Electropherograms may be analyzed to associate features in the data with the presence, absence, or quantities of molecular tags using analysis programs, such as disclosed in Williams et al, U.S. patent publication Ser. No. 2003/0,170,734 A1.

“Specific” or “specificity” in reference to the binding of one molecule to another molecule, such as a binding compound, or probe, for a target analyte or complex, means the recognition, contact, and formation of a stable complex between the probe and target, together with substantially less recognition, contact, or complex formation of the probe with other molecules. In one aspect, “specific” in reference to the binding of a first molecule to a second molecule means that to the extent the first molecule recognizes and forms a complex with another molecules in a reaction or sample, it forms the largest number of the complexes with the second molecule. In one aspect, this largest number is at least fifty percent of all such complexes form by the first molecule. Generally, molecules involved in a specific binding event have areas on their surfaces or in cavities giving rise to specific recognition between the molecules binding to each other. Examples of specific binding include antibody-antigen interactions, enzymesubstrate interactions, formation of duplexes or triplexes among polynucleotides and/or oligonucleotides, receptor-ligand interactions, and the like.

As used herein, the term “spectrally resolvable” in reference to a plurality of fluorescent labels means that the fluorescent emission bands of the labels are sufficiently distinct, i.e. sufficiently non-overlapping, that molecular tags to which the respective labels are attached can be distinguished on the basis of the fluorescent signal generated by the respective labels by standard photodetection systems, e.g. employing a system of band pass filters and photomultiplier tubes, or the like, as exemplified by the systems described in U.S. Pat. Nos. 4,230,558; 4,811,218, or the like, or in Wheeless et al, pgs. 21-76, in Flow Cytometry: Instrumentation and Data Analysis (Academic Press, New York, 1985).

“Substantially identical” in reference to proteins or amino acid sequences of proteins in a family of related proteins that are being compared means either that one protein has an amino acid sequence that is at least fifty percent identical to the other protein or that one protein is an isoform or splice variant of the same gene as the other protein. In one aspect, substantially identical means one protein, or amino acid sequence thereof, is at least eighty percent identical to the other protein, or amino acid sequence thereof.

### DETAILED DESCRIPTION OF THE INVENTION

Methods and systems of the invention permit highly sensitive measurements of membrane-associated analytes of single cells because (i) molecular tags indicative of such analytes are released into a very small volume, e.g. less than 10 nL, and (ii) molecular tags are then separated by electrophoresis and detected. Because of the separation, measurements can be multiplexed and the signals generated have little or no background. As described more fully below, molecular tags may be released from binding compounds attached to membrane-associated analytes in a variety of ways depending on the cleavable linkage employed. In one aspect, reagent pairs are employed that comprise one or more binding compounds and a cleavage probe, where the cleavage probe has a cleavage-inducing moiety that generates an active species for cleaving cleavable linkages within its proximity. Such embodiments are suitable for measuring interactions and complex formation among membrane-associated analytes, such as receptor dimerization or clustering. In another aspect, molecular tags of binding compounds are attached by cleavable linkages that are cleaved by cleavage agents that act generally on, or throughout, the entire, or non-local portions of, reaction mixture. Such cleavage agents, described more fully below, include reducing agents, oxidizing agents, light to cleave photocleavable bonds, and the like. Such embodiments are suitable for measuring the expression of populations of surface molecules, usually without regard to the interaction or distribution of individual species. In a particular embodiment of this aspect, cleavage agents comprise lipophilic photosensitizers molecules that are embedded in the cellular membranes of cells in a sample.

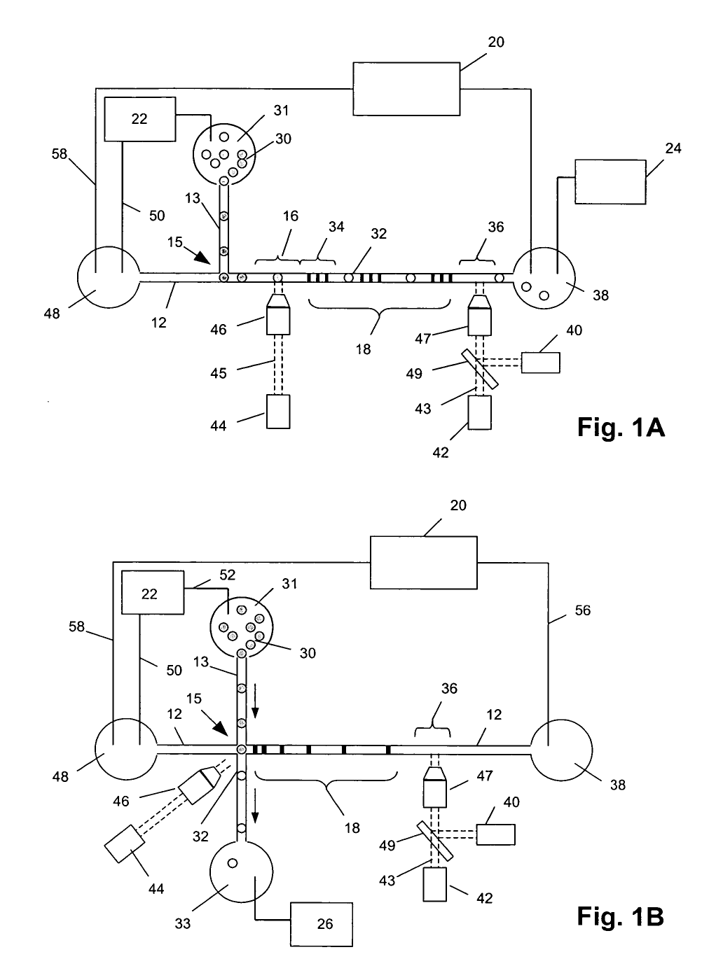

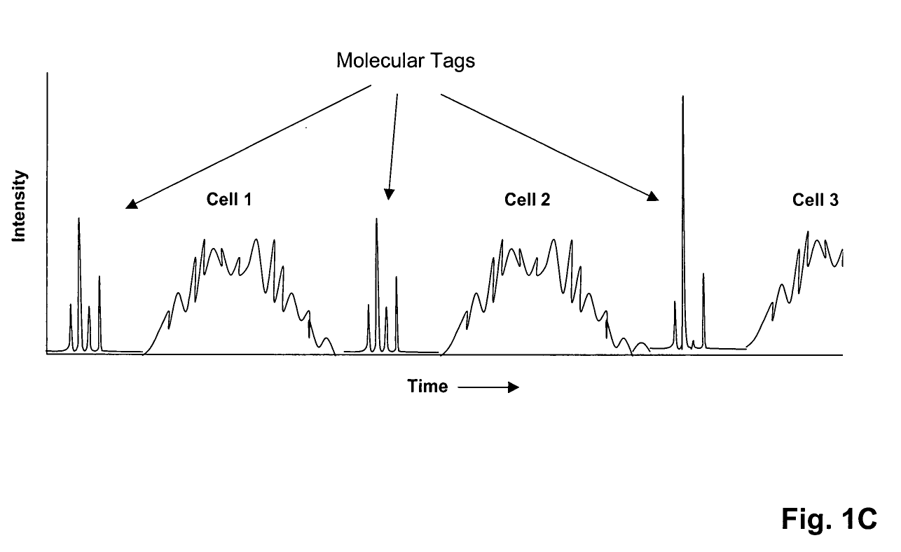

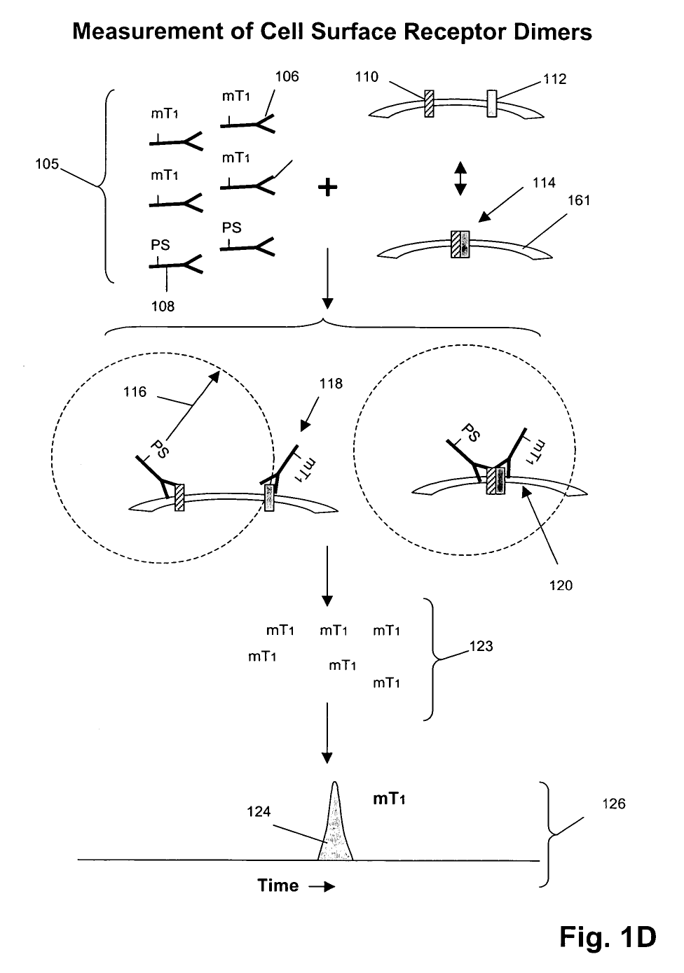

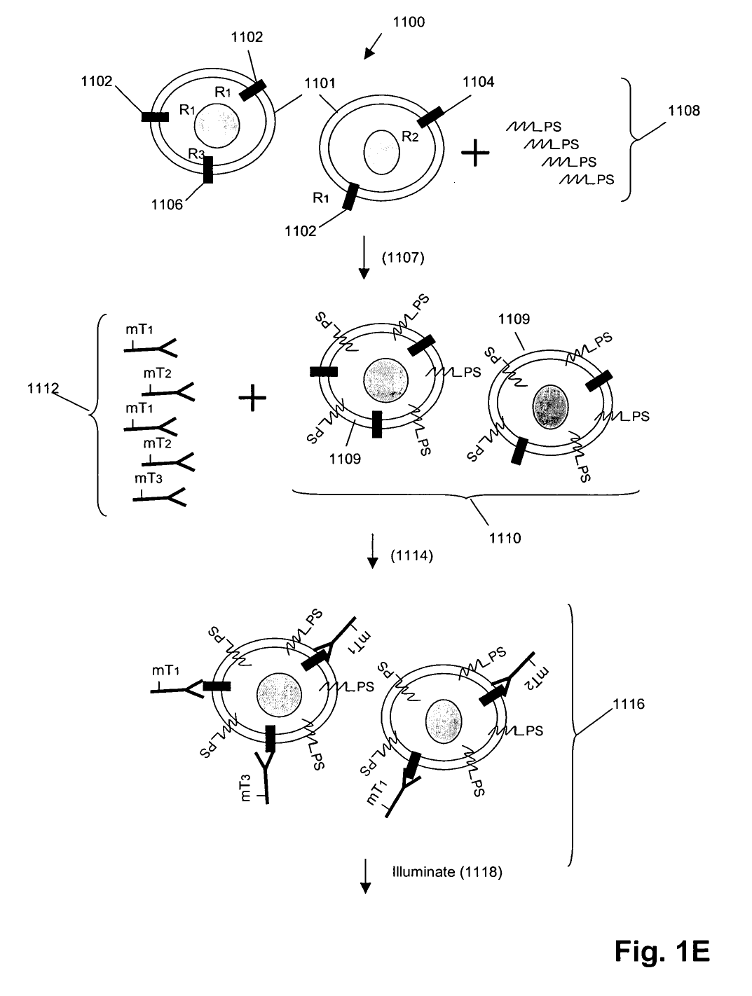

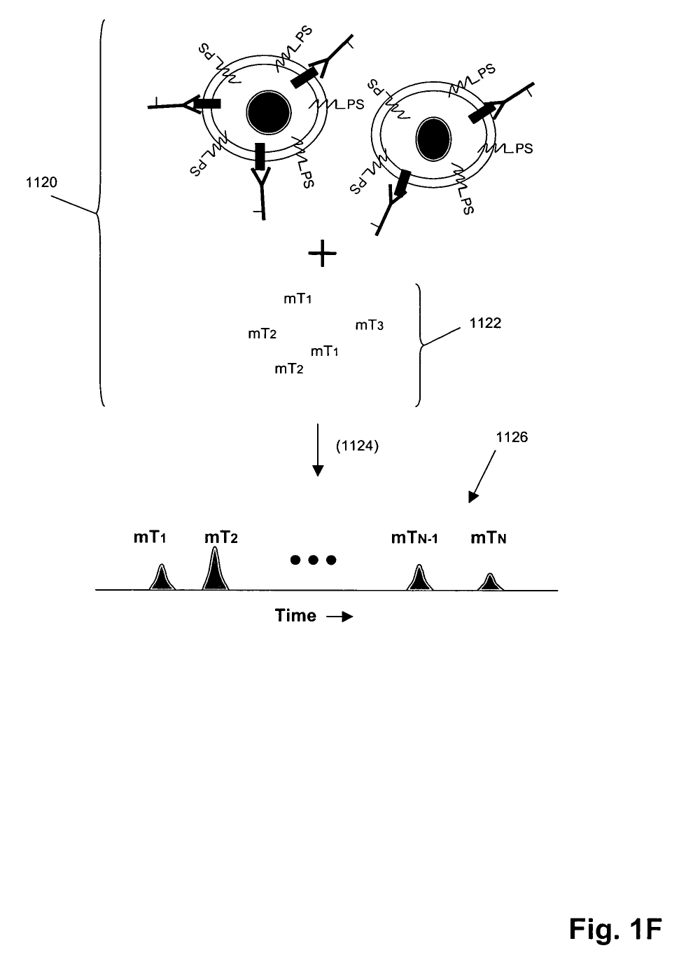

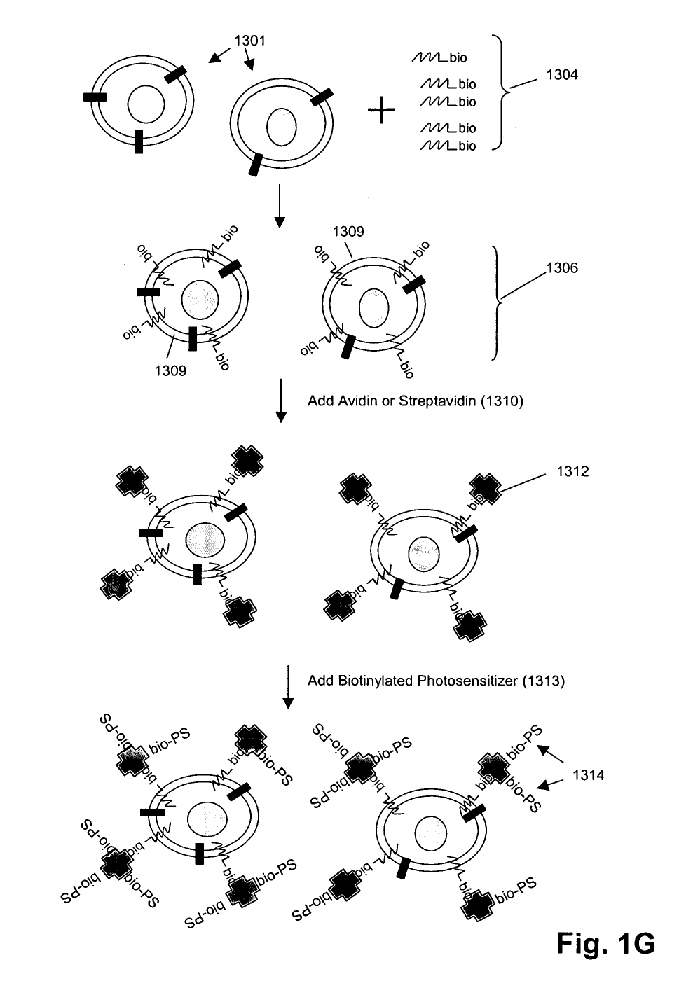

Returning to embodiments employing reagent pairs, surface membrane receptors are labeled with molecular tags and cleaving probes as illustrated in FIG. 1D and disclosed in Singh et al, U.S. Pat. No. 6,627,400, which is incorporated herein by reference. Briefly, in this aspect, the method of the invention is carried out in the following steps: (i) providing a column of buffer solution moving along an electrophoresis channel, the channel having in succession a cleavage zone and a separation zone and having an electrical field collinear with the moving column of buffer solution; (ii) inserting a cell into the moving column of buffer solution upstream of the cleavage zone so that the cell is transported by the moving column of buffer solution through the electrophoresis channel to the cleavage zone, wherein the cell comprises, attached to each of the plurality of surface membrane molecules, (a) a binding composition specific for that surface membrane molecule and having one or more molecular tags, each attached to the binding composition by a cleavable linkage, wherein the molecular tags of different binding compositions have different electrophoretic mobilities, so that distinct peaks are formed upon electrophoretic separation of a mixture of the molecular tags, and (b) a cleaving probe attached to each of the plurality of surface membrane molecules or to a molecule forming complexes therewith, the cleaving probe having a cleavage-inducing moiety having an effective proximity; (iii) activating in the cleavage zone the cleavage-inducing moieties to generate an active species that cleaves cleavable linkages within the effective proximity thereof to release molecular tags from the cell; and (iv) electrophoretically separating and identifying the released molecular tags in the moving column of buffer solution in the separation zone of the electrophoresis channel, to determine the populations of surface membrane molecules of the cell. Operation of reagent pairs is illustrated in FIG. 1D. Mixture (105) comprising binding compounds (106) and cleaving probes (108) are combined with a cell sample containing cells having receptors (110 and 112) that form dimers (114) in its surface membrane (161). When binding compounds and cleaving probes bind to receptors that are not dimerized, then cleavable linkages are outside (118) of the effective proximity (116) of cleavage-inducing moiety (designated as “PS” on the cleaving probe). On the other hand, when a dimer forms, then cleavable linkages are within (120) the effective proximity and molecular tags are released (123). Released molecular tags are then separated electrophoretically to form an electropherogram (126) in which the presence and quantity of dimers may be related to a peak (124).

The above receptor tagging strategies may be implement by the system of the invention illustrated in FIG. 1A. Reservoir (48) is connected to, and in fluid communication with, reservoir (38) by microfluidics channel (12). Reservoir (31) containing cells (30) for analysis and is connect to, and in fluid communication with, channel (12) by sample-injection channel (13). Reservoir (31) contains a buffer compatible with maintaining cells (30) in an integral state. Whenever cells (30) are fixed, the buffer may be identical to an electrophoretic separation buffer, normally present in revervors (38) and (48) and microfluidic channel (12). Whenever cells (30) are non-fixed or living, then revervoir (31) and sample-injection channel (13) contain a physiological buffer that contains sufficient salt concentration to maintain cells (30). An electrical field is established between reservoirs (38) and (48) by high voltage supply (20) which is connected to the reservoirs by electrodes (56) and (58). Reservoirs (48) and (31) each are connected via lines (52) and (50) to regulated pressure source (22) that generates pressure on fluid in the reservoirs to create a controlled flow from each reservoir into channels (12) and (13), respectively. Preferably, pressure source (22) is a pneumatic pressure source conventional in the art, e.g. Unger et al, Science, 288: 113-116 (2000), or the like. Flow from reservoir (31) carries cells (30) from the reservoir into sample-injection channel (13) and to junction (15) where the flows from reservoirs (48) and (31) converge, mix, and then continues in the direction of reservoir (38). Thus, in this embodiment, a column of buffer solution is created at junction (15) that moves within channel (12) in a direction collinear with the electrical field between reservoirs (38) and (48). Preferably, the direction of the electrical field and charge of the molecular tags are selected so that the direction of flow and the electrophoretic path of released molecular tags are collinear and in the same direction, i.e. towards reservoir (38). Reservoir (38) is connected optionally to vacuum source (24) that either works in cooperation with pressure source (22) or works as the sole means for driving fluid from reservoirs (48) and (31) if pressure source (22) is not employed. In this embodiment, cells (30) in the column of moving buffer solution are transported into cleavage zone (16) of channel (12) where they are illuminated by light source (44) whose beam (45) is focused on the cleavage zone by objective (46). Light from light source (44) serves as a direct or indirect cleavage agent to release molecular tags in this embodiment. For example, when molecular tags are attached to binding compounds by photocleavable linkages, then light from light source (44) directly cleaves the molecular tags. When reagent pairs are employed such that cell surface targets have bound both binding compounds and cleaving probes, light from light source (44) cleaves indirectly by activating photosensitizers attached to the cleaving probes, which, in turn, generate active species, usually singlet oxygen, that diffuse to, and react with, adjacent cleavable linkages thereby releasing molecular tags. Illuminated cells (32) and released molecular tags (34) move along separation zone (18) of channel (12) toward reservoir (38). Released molecular tags from each illuminated cell (32) are transported both by the moving column of buffer solution and by electric force, and are separated from one another electrophoretically. Eventually, separated molecular tags pass though detection zone (36). Molecular tags may be detected by a variety of means, including by electrochemical tags, fluorescent dyes, or calorimetric labels. Preferably, molecular tags generate and are detected by an optical signal. More preferably, molecular tags are detected via a fluorescent label, which may be excited to generate fluorescent emissions that collected by a conventional detection system. For example, light source (42) may generate beam (43) that is directed to released molecular tags by optics (47). Fluorescent emissions may be collected by the same optics (47) and directed to dichroic (49) and then to detector (40). After detection, cells (32) and molecular tags pass to reservoir (38) as waste. FIG. 1C illustrates an electropherogram of data that would be obtained with the system of FIG. 1A. There would be signals from molecular tags followed by a signal generated by a cell as it passed the detector. From the peak heights and locations within each set of molecular tag signal, the number of receptors, or receptor dimers, on a cell surface is determined. Programs for analyzing such electropherogram data is well known in the art, e.g. Williams et al, U.S. patent publication Ser. No. 2003/0,170,734.

Another aspect of the system of the invention is illustrated in FIG. 1B, in which similarly numbered items are the same as in FIG. 1A. In this embodiment, cells (30) from reservoir (31) are transported through sample injection channel (13) to junction (15) with channel (12) and then to separate waste reservoir (33). Junction (15) serves as a cleavage zone in that cells (30) are illuminated at that location by light source (44) to directly or indirectly release molecular tags. As in FIG. 1A, released molecular tags are separated electrophoretically along separation zone (18) of channel (12).

Preferably, the sensitivity of the systems of FIGS. 1A and 1B are as follows, based on the assumptions below.

Cell diameter: 10 um

Channel cross section: 50×50 um

Illumination duration: ˜10 sec (scaled flux equivalent to current protocol of 5-min defocused illumination in microtiter plate well)

Diffusion coefficient: ˜1e-5 cm²/sec

Fluorescein LOD on a microfluidic device: 100 pM=1e-10 moles/L

Volume of released molecular tags subject to quiescent diffusion:

L²=1e−5 cm²/sec×10 sec=1e−4 cm²

L=1e−2 cm=1e−1 mm=100 um

V=100×50×50 um=2.5e5 um³=2.5e−4 uL=2.5e2 pL=2.5e−10 L

The above leads to a theoretical LOD as follows:

2.5e−10 L×1e−10 moles/L×6.02e23 molecular tags/mole=1.5e4 molecular tags,

or approximately 10,000 receptors/cell.

Another aspect of the invention is illustrated in FIGS. 1E and 1F. Cells (1100) having cell surface membrane (1101) and receptors (1102, “R1”), (1104, “R2”), and (1106, “R3”)—the membrane-associated analytes—are incubated with lipophilic photosensitizers (1108) to form (1107) photosensitizer-treated membranes (1109), which are part of intact cells (1110). Binding compounds (1112) having molecular tags (“mTₖ”) attached are combined with photosensitizer-treated membranes (1109) under conditions that permit specific binding of binding compounds (1112) to their respective target membrane-associated analytes. Reaction mixture (1116) is then illuminated (1118) with a light of wavelength and intensity to excite the membrane-bound photosensitizers to generated singlet oxygen which, in turn, cleaves the molecular tags (1122) from the binding compounds, shown in FIG. 1B. Molecular tags (1122) are then separated (1124) from reaction mixture (1120) and identified in separation profile (1126).

### Assay Formats

Membrane-associated analytes may be detected in assays having homogeneous formats or a non-homogeneous, i.e. heterogeneous, formats. In a homogeneous format, no step is required to separate binding compounds specifically bound to target complexes from unbound binding compounds. That is, binding compositions can be mixed with a cell sample and directly loaded into a sample reservoir of a microfluidics device. In a preferred embodiment, homogeneous formats employ reagent pairs comprising (i) one or more binding compounds with releasable molecular tags and (ii) at least one cleaving probe that is capable of generating an active species that reacts with and releases molecular tags within an effective proximity of the cleaving probe.

Intracellular complexes may also be detected by assays employing a heterogeneous format. Heterogeneous techniques normally involve a separation step, where intracellular complexes having binding compounds specifically bound are separated from unbound binding compounds, and optionally, other cellular components, such as intracellular complexes, proteins, membrane fragments, and the like. Separation can be achieved in a variety of ways, each employing a reagent bound to a solid support that distinguishes between complex-bound and unbound binding compounds. The solid support may be a vessel wall, e.g., microtiter well plate well, capillary, plate, slide, beads, including magnetic beads, liposomes, or the like. The primary characteristics of the solid support are that it (1) permits segregation of the bound and unbound binding compounds and (2) does not interfere with the formation of the binding complex, nor the other operations in the determination of the intracellular complex.

When releasable molecular tags are employed in a heterogeneous format, a releasing agent, i.e. a cleavaging agent, need not be proximity dependent, since target intracellular complexes are separated from unbound binding compounds. Therefore, a larger variety of cleavage protocols can be used to release molecular tags. Cleavage may still be carried out using a sensitizer, as described below, but it may also employ various types of chemical, photochemical, or enzymatic cleavage of a variety of cleavable linking groups, such as are known in the art. As described more fully below, non-limiting examples of chemically cleavable linkages include disulfides (cleavable by reduction, typically using dithiothreitol), azo groups (cleavable with dithionate), sulfones (cleavable with basic phosphate, with or without dithiothreitol), glycols, cleavable by periodate, and esters, cleavable by hydrolysis. Photolabile linkers include, for example, azo linkages and o-nitrobenzyl ethers. In particular, disulfide bonds are preferred as cleavable linkages whenever heterogeneous formats are employed.

With detection using molecular tags in a heterogeneous format, after washing, a support may be combined with a solvent into which the molecular tags are to be released. Depending on the nature of the cleavable bond and the method of cleavage, the solvent may include any additional reagents for the cleavage. Where reagents for cleavage are not required, the solvent conveniently may be a separation buffer, e.g. an electrophoretic separation medium. For example, where the cleavable linkage is photolabile, the medium may be irradiated with light of appropriate wavelength to release the molecular tags into the buffer.

In either format, if the assay reaction conditions interfere with the separation technique employed, it may be necessary to remove, or exchange, the assay reaction buffer prior to cleavage and separation of the molecular tags. For example, in some embodiments, assay conditions include salt concentrations (e.g. required for specific binding) that degrade separation performance when molecular tags are separated on the basis of electrophoretic mobility.

Guidance for selecting cleaving agents, molecular tags, cleavable linkages, and other components for homogeneous or heterogeneous assay formats is disclosed in the following references: International patent publications WO 00/66607; WO 01/83502; WO 02/95356; WO 03/06947; and U.S. Pat. Nos. 6,322,980 and 6,514,700.

### Binding Compounds and Compositions

As mentioned above, an aspect of the invention includes providing mixtures of pluralities of different binding compounds (such mixture being referred to herein as “binding compositions”), wherein each different binding compound has one or more molecular tags attached through cleavable linkages. The nature of the binding compound, cleavable linkage and molecular tag may vary widely. A binding compound may comprise an antibody binding composition, an antibody, a peptide, a peptide or non-peptide ligand for a cell surface receptor, a protein, an oligonucleotide, an oligonucleotide analog, such as a peptide nucleic acid, a lectin, or any other molecular entity that is capable of specific binding or stable complex formation with an analyte of interest, such as a complex of proteins. In one aspect, a binding compound, which can be represented by the formula below, comprises one or more molecular tags attached to a binding moiety.

B-(L-E)ₖ

wherein B is binding moiety; L is a cleavable linkage; and E is a molecular tag. In homogeneous assays, cleavable linkage, L, may be an oxidation-labile linkage, and more preferably, it is a linkage that may be cleaved by singlet oxygen. The moiety “−(L−E)k” indicates that a single binding compound may have multiple molecular tags attached via cleavable linkages. In one aspect, k is an integer greater than or equal to one, but in other embodiments, k may be greater than several hundred, e.g. 100 to 500, or k is greater than several hundred to as many as several thousand, e.g. 500 to 5000. Usually each of the plurality of different types of binding compound has a different molecular tag, E. Cleavable linkages, e.g. oxidation-labile linkages, and molecular tags, E, are attached to B by way of conventional chemistries.

Preferably, B is an antibody binding composition produced by conventional techniques.

Cleavable linkage, L, can be virtually any chemical linking group that may be cleaved under conditions that do not degrade the structure or affect detection characteristics of the released molecular tag, E. Whenever a cleaving probe is used in a homogeneous assay format, cleavable linkage, L, is cleaved by a cleavage agent generated by the cleaving probe that acts over a short distance so that only cleavable linkages in the immediate proximity of the cleaving probe are cleaved. Typically, such an agent must be activated by making a physical or chemical change to the reaction mixture so that the agent produces a short lived active species that diffuses to a cleavable linkage to effect cleavage. In a homogeneous format, the cleavage agent is preferably attached to a binding moiety, such as an antibody, that targets prior to activation the cleavage agent to a particular site in the proximity of a binding compound with releasable molecular tags. In such embodiments, a cleavage agent is referred to herein as a “cleavage-inducing moiety,” which is discussed more fully below.

In a non-homogeneous format, because specifically bound binding compounds are separated from unbound binding compounds, a wider selection of cleavable linkages and cleavage agents are available for use. Cleavable linkages may not only include linkages that are labile to reaction with a locally acting reactive species, such as hydrogen peroxide, singlet oxygen, or the like, but also linkages that are labile to agents that operate throughout a reaction mixture, such as base-labile linkages, photocleavable linkages, linkages cleavable by reduction, linkages cleaved by oxidation, acid-labile linkages, peptide linkages cleavable by specific proteases, and the like. References describing many such linkages include Greene and Wuts, Protective Groups in Organic Synthesis, Second Edition (John Wiley & Sons, New York, 1991); Hermanson, Bioconjugate Techniques (Academic Press, New York, 1996); and Still et al, U.S. Pat. No. 5,565,324. Exemplary cleavable linkages are illustrated in Table I.

TABLE I

| Linking Group | Cleavage Reagent |
|---|---|
| silyl | fluoride or acid |
| A | hv |
| B | Ce(NH₄)₂(NO₃)₆ |
| —NCO₂— | HO⁻, H⁺, or LiAlH₄ |
| C | O₃, OsO₄/IO₄⁻, or KMnO₄ |
| D | 1) O₂ or Br₂, MeOH |
| | 2) H₃O⁺ |
| —Si— | oxidation, H⁺, Br₂, Cl₂, etc. |
| E | H₃O⁺ |
| F | H₃O⁺ |
| G | F⁻ or H⁺ |
| H, where x is a keto, ester, amide, | base, HO⁻ |
| NO₂, sulfide, sulfoxide, sulfone, and | |
| related electron withdrawing groups. | |
| I | H₃O⁺ or reduction (e.g. Li/NH₃) |
| J | (Ph₃P)₃RhCl(H) |
| K | Li, Mg, or BuLi |
| M | Hg⁺² |
| N, where x is halogen or pseudo- | Zn or Mg |
| halogen | |
| O | oxidation (e.g. Pb(OAc)₄ or H₃IO₆) |
| P, where X is a electron withdrawing | base |
| group | |

Illustrative cleavable linking groups and cleavage reagents (L) shows the point of attachment of the molecular tag (E).

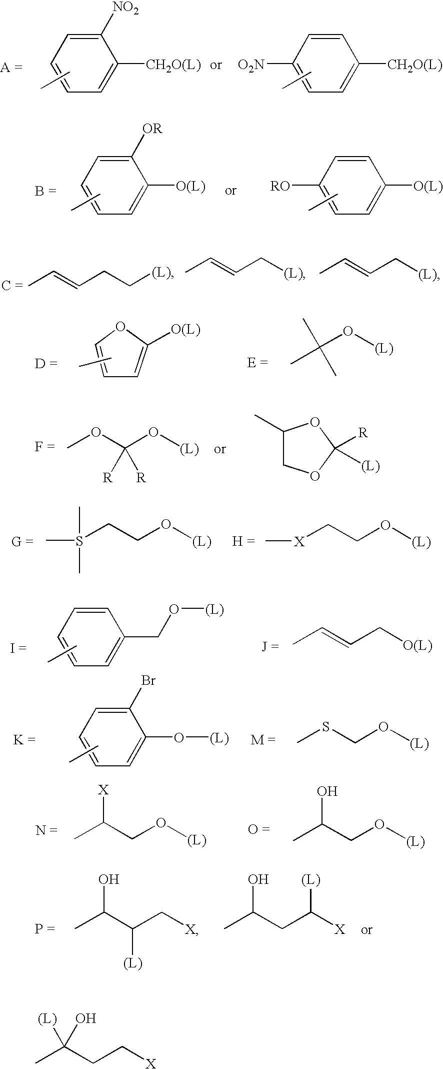

In one aspect, commercially available cleavable reagent systems may be employed with the invention. For example, a disulfide linkage may be introduced between an antibody binding composition and a molecular tag using a heterofunctional agent such as N-succinimidyl 3-(2-pyridldithio)propionate (SPDP), succinimidyloxycarbonyl-α-methyl-α-(2-pyridyldithio)toluene (SMPT), or the like, available from vendors such as Pierce Chemical Company (Rockford, Ill.). Disulfide bonds introduced by such linkages can be broken by treatment with a reducing agent, such as dithiothreitol (DTT), dithioerythritol (DTE), 2-mercaptoethanol, sodium borohydride, or the like. Typical concentrations of reducing agents to effect cleavage of disulfide bonds are in the range of from 10 to 100 mM. An oxidatively labile linkage may be introduced between an antibody binding composition and a molecular tag using the homobifunctional NHS ester cross-linking reagent, disuccinimidyl tartarate (DST)(available from Pierce) that contains central cis-diols that are susceptible to cleavage with sodium periodate (e.g., 15 mM periodate at physiological pH for 4 hours). Linkages that contain esterified spacer components may be cleaved with strong nucleophilic agents, such as hydroxylamine, e.g. 0.1 N hydroxylamine, pH 8.5, for 3-6 hours at 37° C. Such spacers can be introduced by a homobifunctional cross-linking agent such as ethylene glycol bis(succinimidylsuccinate)(EGS) available from Pierce (Rockford, Ill.). A base labile linkage can be introduced with a sulfone group. Homobifunctional cross-linking agents that can be used to introduce sulfone groups in a cleavable linkage include bis[2-(succinimidyloxycarbonyloxy)ethyl]sulfone (BSOCOES), and 4,4-difluoro-3,3-dinitrophenylsulfone (DFDNPS). Exemplary basic conditions for cleavage include 0.1 M sodium phosphate, adjusted to pH 11.6 by addition of Tris base, containing 6 M urea, 0.1% SDS, and 2 mM DTT, with incubation at 37° C. for 2 hours. Photocleavable linkages include those disclosed in Rothschild et al, U.S. Pat. No. 5,986,076.

When L is oxidation labile, L may be a thioether or its selenium analog; or an olefm, which contains carbon-carbon double bonds, wherein cleavage of a double bond to an oxo group, releases the molecular tag, E. Illustrative thioether bonds are disclosed in Willner et al, U.S. Pat. No. 5,622,929 which is incorporated by reference. Illustrative olefins include vinyl sulfides, vinyl ethers, enamines, imines substituted at the carbon atoms with an α-methine (CH, a carbon atom having at least one hydrogen atom), where the vinyl group may be in a ring, the heteroatom may be in a ring, or substituted on the cyclic olefinic carbon atom, and there will be at least one and up to four heteroatoms bonded to the olefinic carbon atoms. The resulting dioxetane may decompose spontaneously, by heating above ambient temperature, usually below about 75° C., by reaction with acid or base, or by photo-activation in the absence or presence of a photosensitizer. Such linkages and reactions are described in the following exemplary references: U.S. Pat. Nos. 5,756,726; 5,800,999; and 5,886,238.

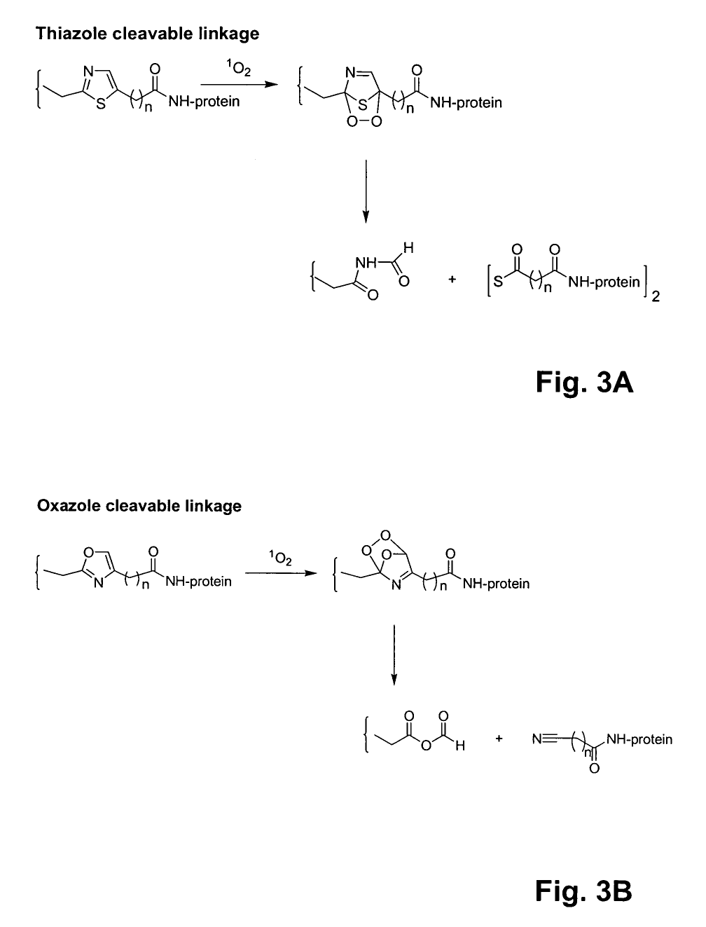

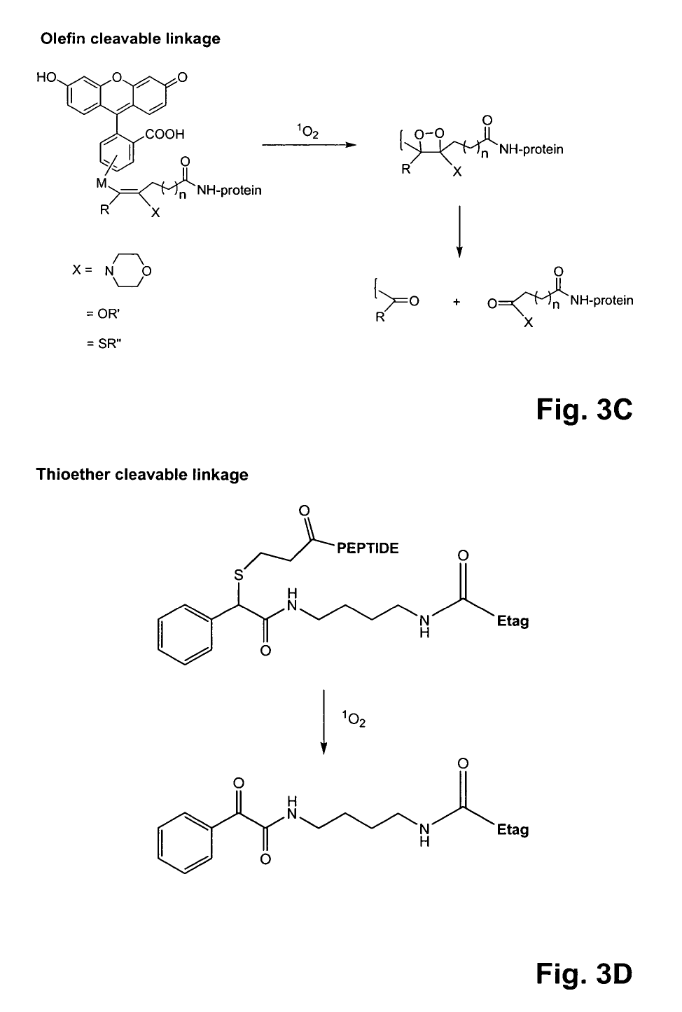

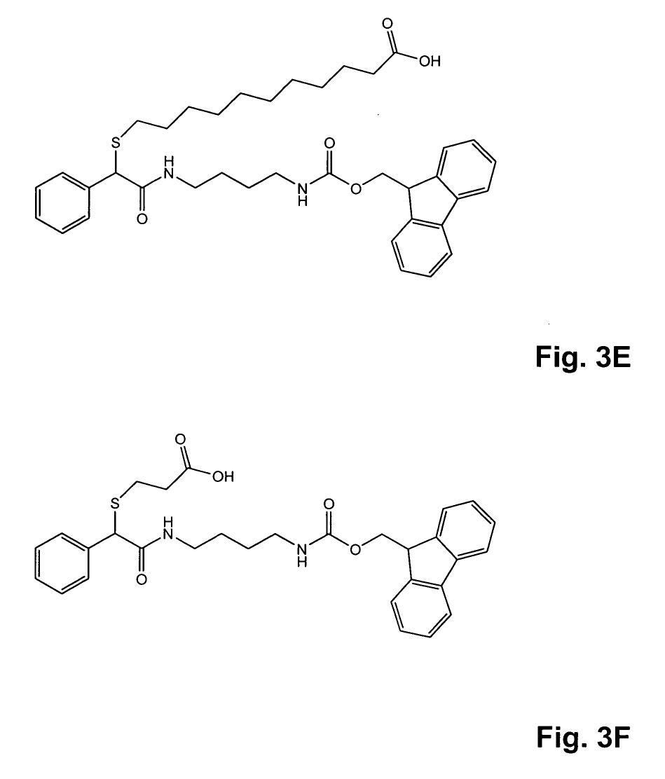

Exemplary cleavable linkages and their cleavage products are illustrated in FIGS. 3 A-F. The thiazole cleavable linkage, “—CH₂-thiazole-(CH₂)ₙ—C(═O)—NH-protein,” shown in FIG. 3A, results in an molecular tag with the moiety “—CH₂—C(═O)—NH—CHO.” Preferably, n is in the range of from 1 to 12, and more preferably, from 1 to 6. The oxazole cleavable linkage, “—CH₂-oxazole-(CH₂) shown in FIG. 3B, results in an molecular tag with the moiety “—CH₂—C(═O)O—CHO.” An olefin cleavable linkage (FIG. 3C) is shown in connection with the binding compound embodiment “B-L-M-D,” described above and with D being a detection moiety, such as a fluorescein dye. The olefin cleavable linkage may be employed in other embodiments also. Cleavage of the illustrated olefin linkage results in an molecular tag of the form: “R—(C═O)-M-D,” where “R” may be any substituent within the general description of the molecular tags, E, provided above. Preferably, R is an electron-donating group, e.g. Ullman et al, U.S. Pat. No. 6,251,581; Smith and March, March's Advanced Organic Chemistry: Reactions, Mechanisms, and Structure, 5^{th} Edition (Wiley-Interscience, New York, 2001); and the like. More preferably, R is an electron-donating group having from 1-8 carbon atoms and from 0 to 4 heteroatoms selected from the group consisting of O, S, and N. In further preference, R is —N(Q)₂, —OQ, p-[C₆H₄N(Q)₂], furanyl, n-alkylpyrrolyl, 2-indolyl, or the like, where Q is alkyl or aryl. In further reference to the olefin cleavable linkage of FIG. 3C, substituents “X” and “R” are equivalent to substituents “X” and “Y” of the above formula describing cleavable linkage, L. In particular, X in FIG. 3C is preferably morpholino, —OR′, or —SR″, where R′ and R″ are aliphatic, aromatic, alicyclic or heterocyclic having from 1 to 8 carbon atoms and 0 to 4 heteroatoms selected from the group consisting of O, S. and N. A preferred thioether cleavable linkage is illustrated in FIG. 3D having the form “—(CH₂)₂—S—CH(C₆H₅)C(═O)NH—(CH₂)ₙ—NH—,” wherein n is in the range of from 2 to 12, and more preferably, in the range of from 2 to 6. Thioether cleavable linkages of the type shown in FIG. 3D may be attached to binding moieties, T, and molecular tags, E, by way of precursor compounds shown in FIGS. 3E and 3F. To attach to an amino group of a binding moiety, T, the terminal hydroxyl is converted to an NHS ester by conventional chemistry. After reaction with the amino group and attachment, the Fmoc protection group is removed to produce a free amine which is then reacted with an NHS ester of the molecular tag.

Molecular tag, E, in the present invention may comprise an electrophoric tag as described in the following references when separation of pluralities of molecular tags are carried out by gas chromatography or mass spectrometry: Zhang et al, Bioconjugate Chem., 13: 1002-1012 (2002); Giese, Anal. Chem., 2: 165-168 (1983); and U.S. Pat. Nos. 4,650,750; 5,360,819; 5,516,931; 5,602,273; and the like.

Molecular tag, E, is preferably a water-soluble organic compound that is stable with respect to the active species, especially singlet oxygen, and that includes a detection or reporter group. Otherwise, E may vary widely in size and structure. In one aspect, E has a molecular weight in the range of from about 50 to about 2500 daltons, more preferably, from about 50 to about 1500 daltons. Preferred structures of E are described more fully below. E may comprise a detection group for generating an electrochemical, fluorescent, or chromogenic signal. In embodiments employing detection by mass, E may not have a separate moiety for detection purposes. Preferably, the detection group generates a fluorescent signal.

Molecular tags within a plurality are selected so that each has a unique separation characteristic and/or a unique optical property with respect to the other members of the same plurality. In one aspect, the chromatographic or electrophoretic separation characteristic is retention time under set of standard separation conditions conventional in the art, e.g. voltage, column pressure, column type, mobile phase, electrophoretic separation medium, or the like. In another aspect, the optical property is a fluorescence property, such as emission spectrum, fluorescence lifetime, fluorescence intensity at a given wavelength or band of wavelengths, or the like. Preferably, the fluorescence property is fluorescence intensity. For example, each molecular tag of a plurality may have the same fluorescent emission properties, but each will differ from one another by virtue of a unique retention time. On the other hand, or two or more of the molecular tags of a plurality may have identical migration, or retention, times, but they will have unique fluorescent properties, e.g. spectrally resolvable emission spectra, so that all the members of the plurality are distinguishable by the combination of molecular separation and fluorescence measurement.

Preferably, released molecular tags are detected by electrophoretic separation and the fluorescence of a detection group. In such embodiments, molecular tags having substantially identical fluorescence properties have different electrophoretic mobilities so that distinct peaks in an electropherogram are formed under separation conditions. Preferably, pluralities of molecular tags of the invention are separated by conventional capillary electrophoresis apparatus, either in the presence or absence of a conventional sieving matrix. Exemplary capillary electrophoresis apparatus include Applied Biosystems (Foster City, Calif.) models 310, 3100 and 3700; Beckman (Fullerton, Calif.) model P/ACE MDQ; Amersham Biosciences (Sunnyvale, Calif.) MegaBACE 1000 or 4000; SpectruMedix genetic analysis system; and the like. Electrophoretic mobility is proportional to q/M^{2/3}, where q is the charge on the molecule and M is the mass of the molecule. Desirably, the difference in mobility under the conditions of the determination between the closest electrophoretic labels will be at least about 0.001, usually 0.002, more usually at least about 0.01, and may be 0.02 or more. Preferably, in such conventional apparatus, the electrophoretic mobilities of molecular tags of a plurality differ by at least one percent, and more preferably, by at least a percentage in the range of from 1 to 10 percent.

In one aspect, molecular tag, E, is (M, D), where M is a mobility-modifying moiety and D is a detection moiety. The notation “(M, D)” is used to indicate that the ordering of the M and D moieties may be such that either moiety can be adjacent to the cleavable linkage, L. That is, “B-L-(M, D)” designates binding compound of either of two forms: “B-L-M-D” or “B-L-D-M.”

Detection moiety, D, may be a fluorescent label or dye, a chromogenic label or dye, an electrochemical label, or the like. Preferably, D is a fluorescent dye. Exemplary fluorescent dyes for use with the invention include water-soluble rhodamine dyes, fluoresceins, 4,7-dichlorofluoresceins, benzoxanthene dyes, and energy transfer dyes, disclosed in the following references: Handbook of Molecular Probes and Research Reagents, 8^{th} ed., (Molecular Probes, Eugene, 2002); Lee et al, U.S. Pat. No. 6,191,278; Lee et al, U.S. Pat. No. 6,372,907; Menchen et al, U.S. Pat. No. 6,096,723; Lee et al, U.S. Pat. No. 5,945,526; Lee et al, Nucleic Acids Research, 25: 2816-2822 (1997); Hobb, Jr., U.S. Pat. No. 4,997,928; Khanna et al., U.S. Pat. No. 4,318,846; and the like. Preferably, D is a fluorescein or a fluorescein derivative.

The size and composition of mobility-modifying moiety, M, can vary from a bond to about 100 atoms in a chain, usually not more than about 60 atoms, more usually not more than about 30 atoms, where the atoms are carbon, oxygen, nitrogen, phosphorous, boron and sulfur. Generally, when other than a bond, the mobility-modifying moiety has from about 0 to about 40, more usually from about 0 to about 30 heteroatoms, which in addition to the heteroatoms indicated above may include halogen or other heteroatom. The total number of atoms other than hydrogen is generally fewer than about 200 atoms, usually fewer than about 100 atoms. Where acid groups are present, depending upon the pH of the medium in which the mobility-modifying moiety is present, various cations may be associated with the acid group. The acids may be organic or inorganic, including carboxyl, thionocarboxyl, thiocarboxyl, hydroxamic, phosphate, phosphite, phosphonate, phosphinate, sulfonate, sulfinate, boronic, nitric, nitrous, etc. For positive charges, substituents include amino (includes ammonium), phosphonium, sulfonium, oxonium, etc., where substituents are generally aliphatic of from about 1-6 carbon atoms, the total number of carbon atoms per heteroatom, usually be less than about 12, usually less than about 9. The side chains include amines, ammonium salts, hydroxyl groups, including phenolic groups, carboxyl groups, esters, amides, phosphates, heterocycles. M may be a homo-oligomer or a hetero-oligomer, having different monomers of the same or different chemical characteristics, e.g., nucleotides and amino acids.

Extensive guidance can be found in the literature for covalently linking molecular tags to binding compounds, such as antibodies, e.g. Hermanson, Bioconjugate Techniques, (Academic Press, New York, 1996), and the like. In one aspect of the invention, one or more molecular tags are attached directly or indirectly to common reactive groups on a binding compound. Common reactive groups include amine, thiol, carboxylate, hydroxyl, aldehyde, ketone, and the like, and may be coupled to molecular tags by commercially available cross-linking agents, e.g. Hermanson (cited above); Haugland, Handbook of Fluorescent Probes and Research Products, Ninth Edition (Molecular Probes, Eugene, Oreg., 2002). In one embodiment, an NHS-ester of a molecular tag is reacted with a free amine on the binding compound.

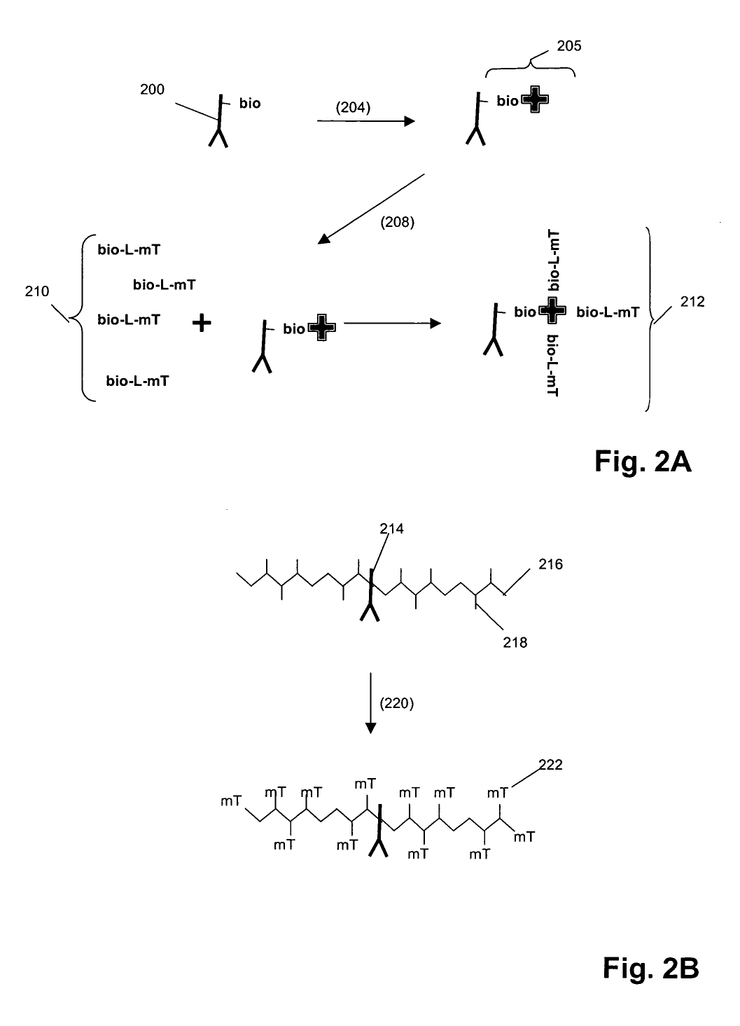

In another embodiment illustrated in FIG. 2A, binding compounds comprise a biotinylated antibody (200) as a binding moiety. Molecular tags are attached to binding moiety (200) by way of avidin or streptavidin bridge (206). Preferably, in operation, binding moiety (200) is first reacted with a target complex, after which avidin or streptavidin is added (204) to form antibody-biotin-avidin complex (205). To such complexes (205) are added (208) biotinylated molecular tags (210) to form binding compound (212).

In still another embodiment illustrated in FIG. 2B, binding compounds comprise an antibody (214) derivatized with a multi-functional moiety (216) that contains multiple functional groups (218) that are reacted (220) molecular tag precursors to give a final binding compound having multiple molecular tags (222) attached. Exemplary multi-functional moieties include aminodextran, and like materials.

Once each of the binding compounds is separately derivatized by a different molecular tag, it is pooled with other binding compounds to form a plurality of binding compounds. Usually, each different kind of binding compound is present in a composition in the same proportion; however, proportions may be varied as a design choice so that one or a subset of particular binding compounds are present in greater or lower proportion depending on the desirability or requirements for a particular embodiment or assay. Factors that may affect such design choices include, but are not limited to, antibody affinity and avidity for a particular target, relative prevalence of a target, fluorescent characteristics of a detection moiety of a molecular tag, and the like.

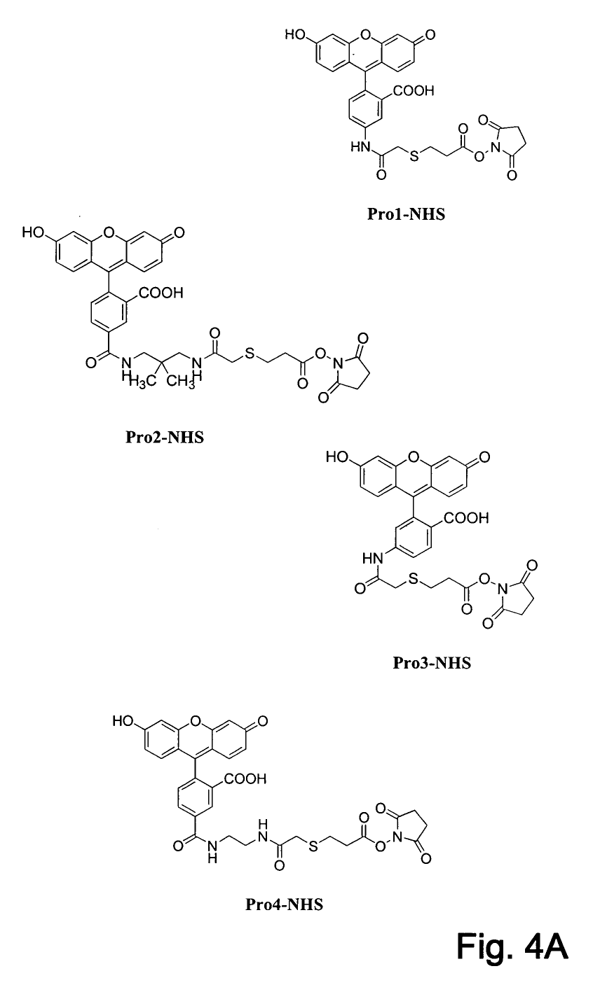

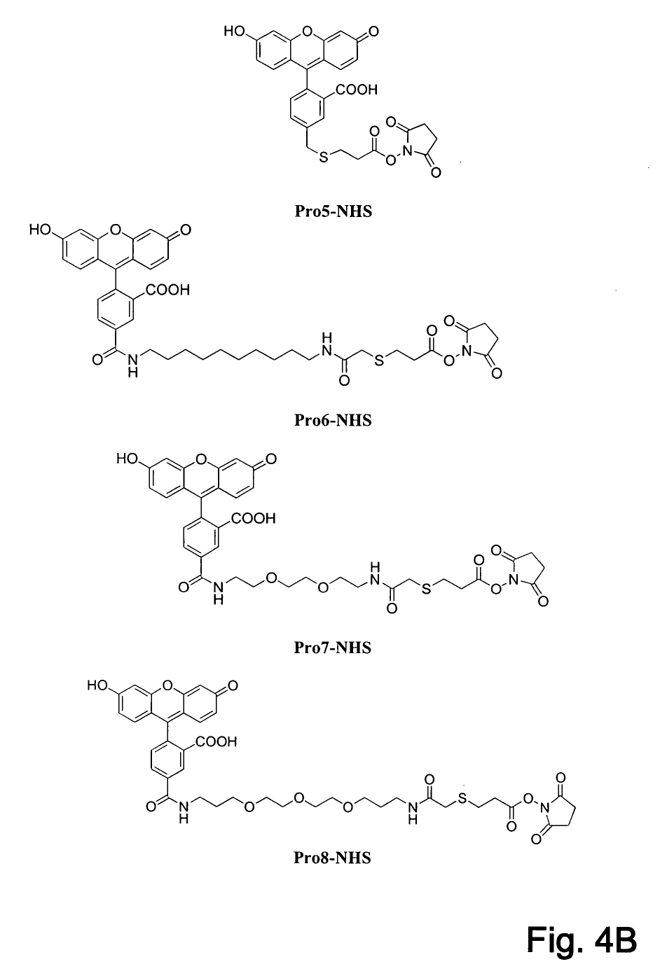

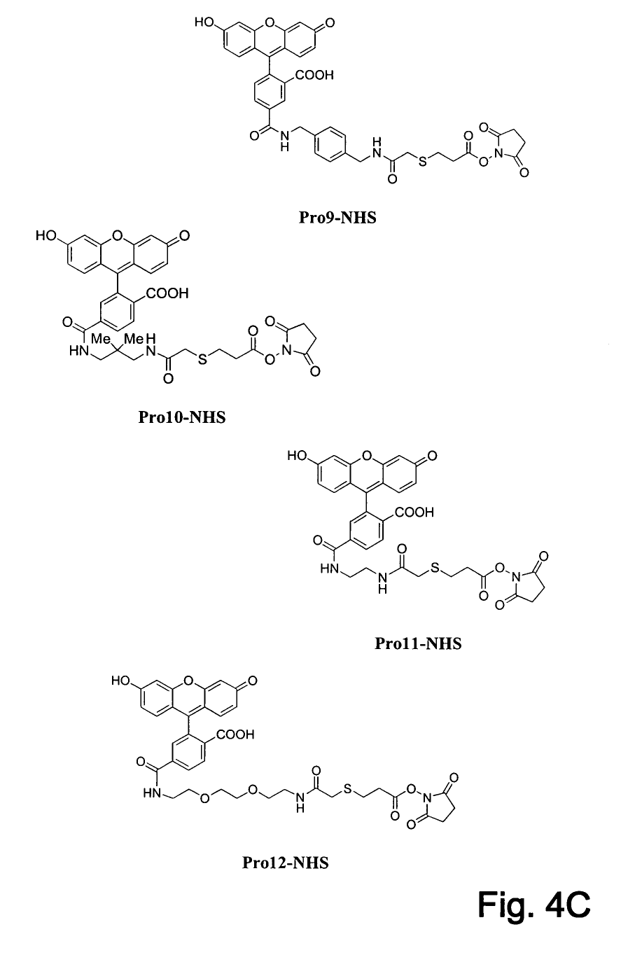

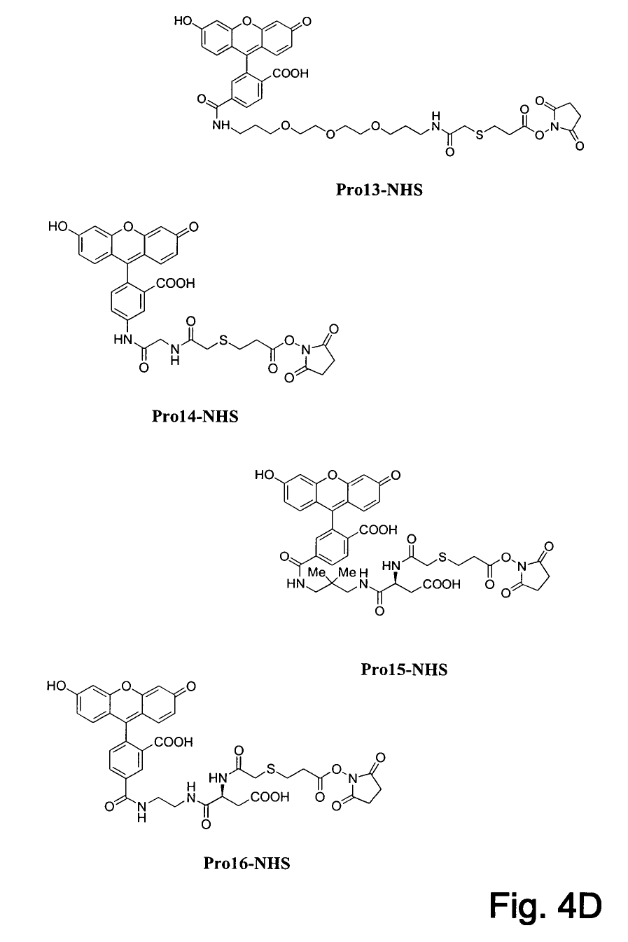

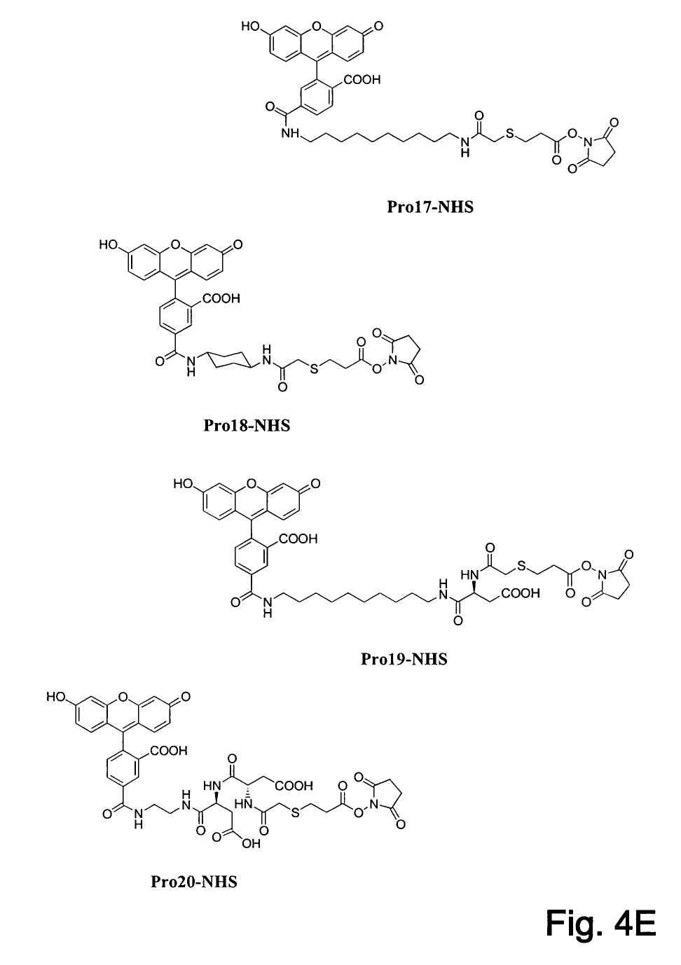

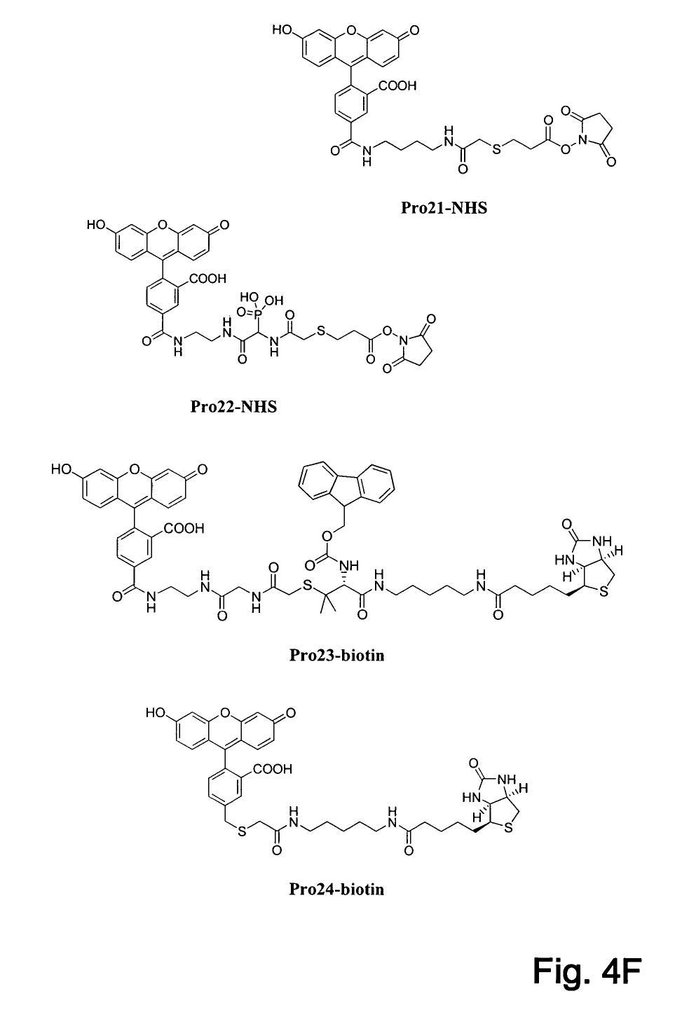

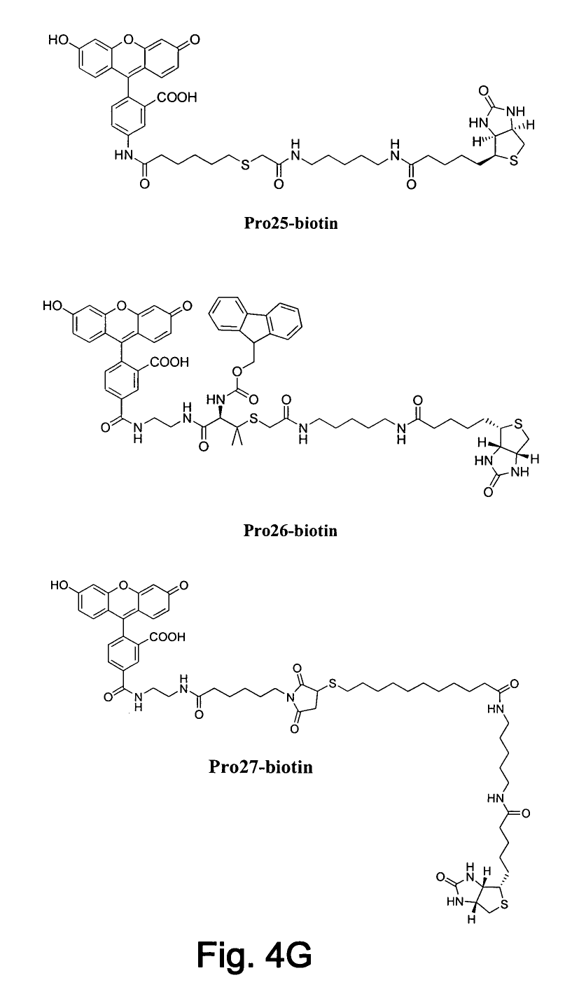

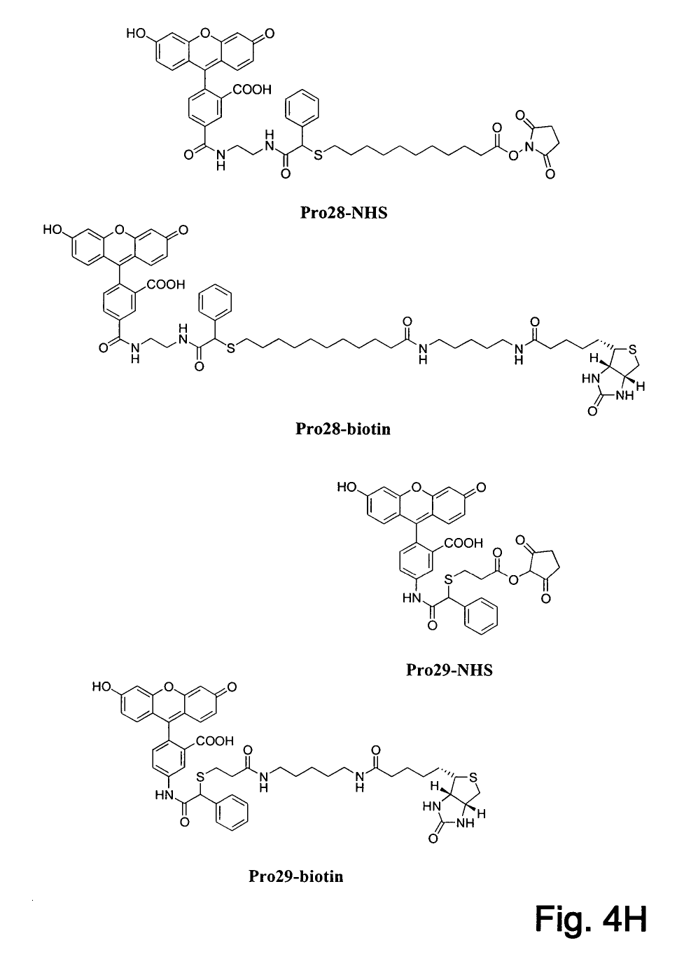

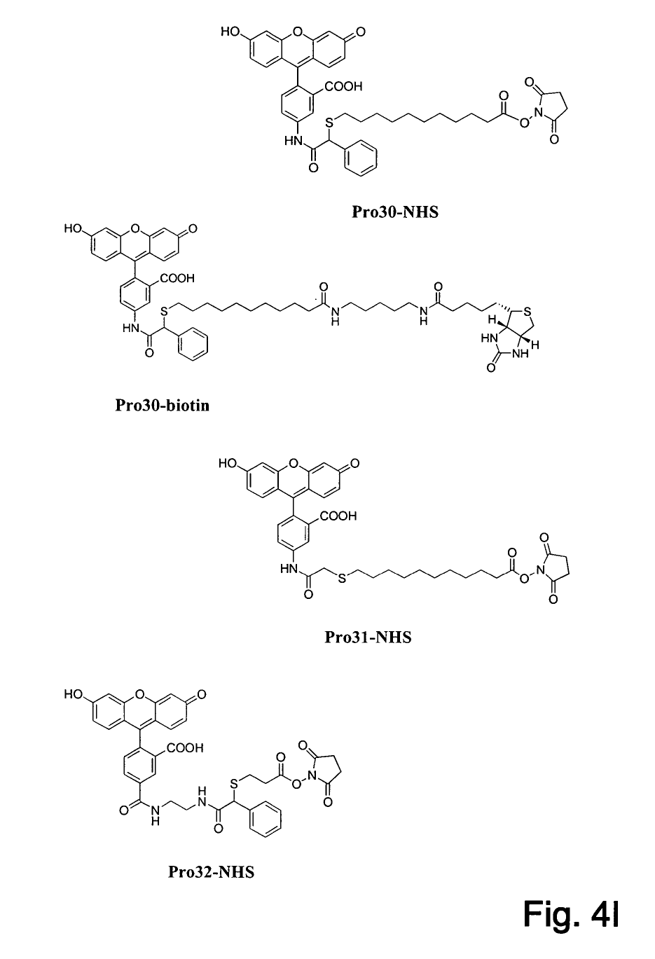

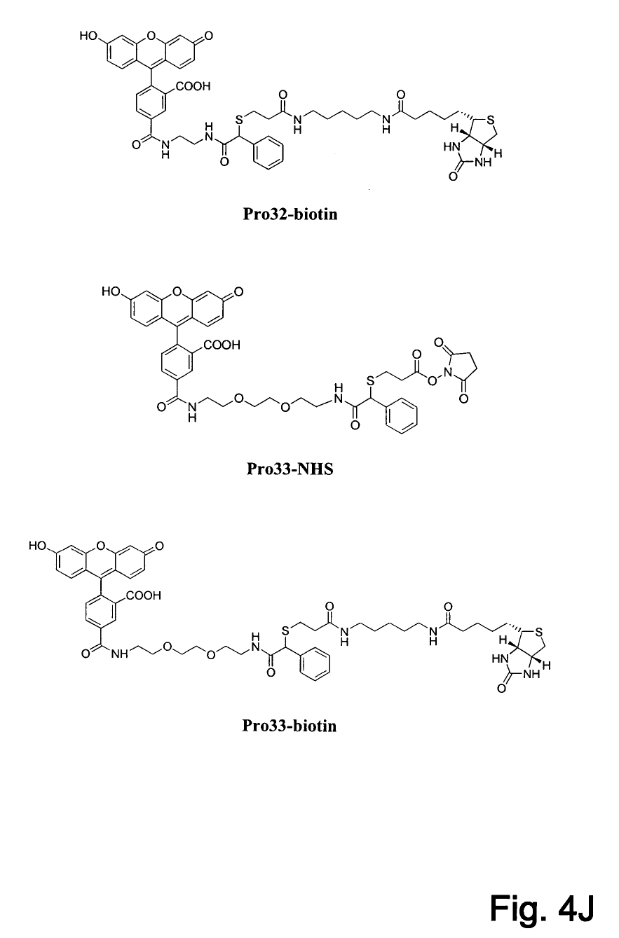

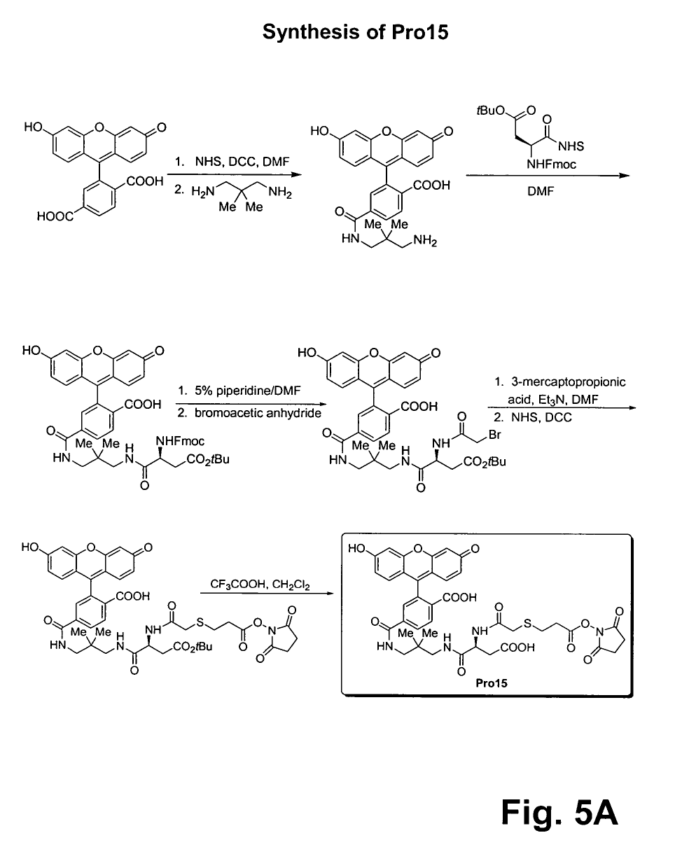

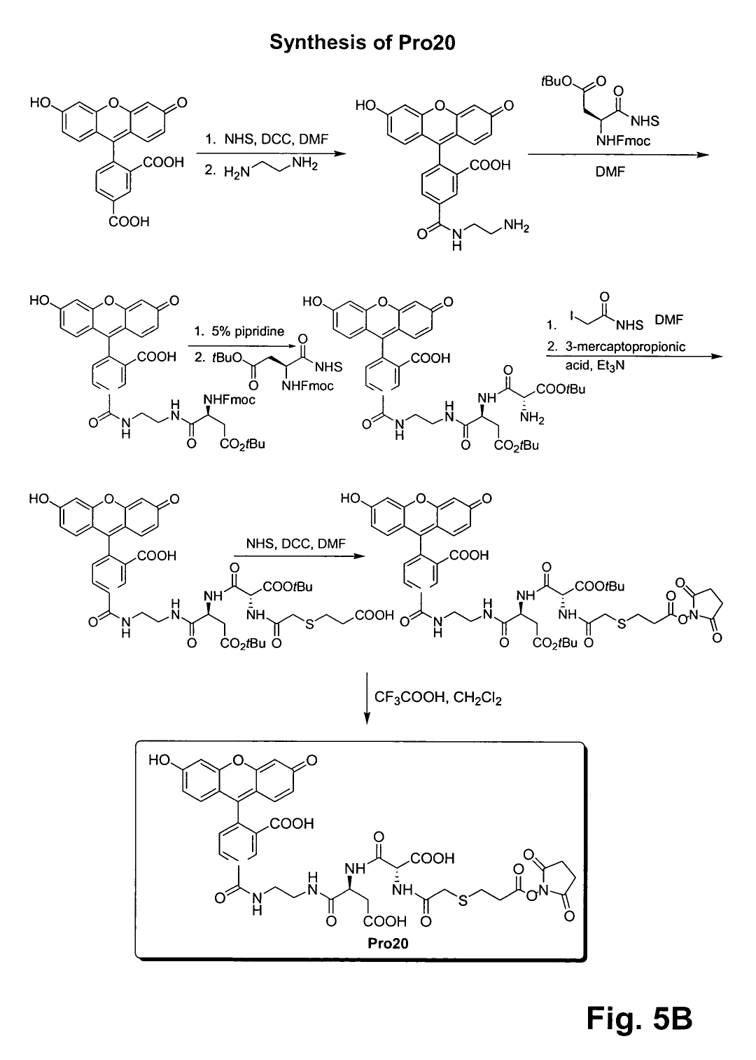

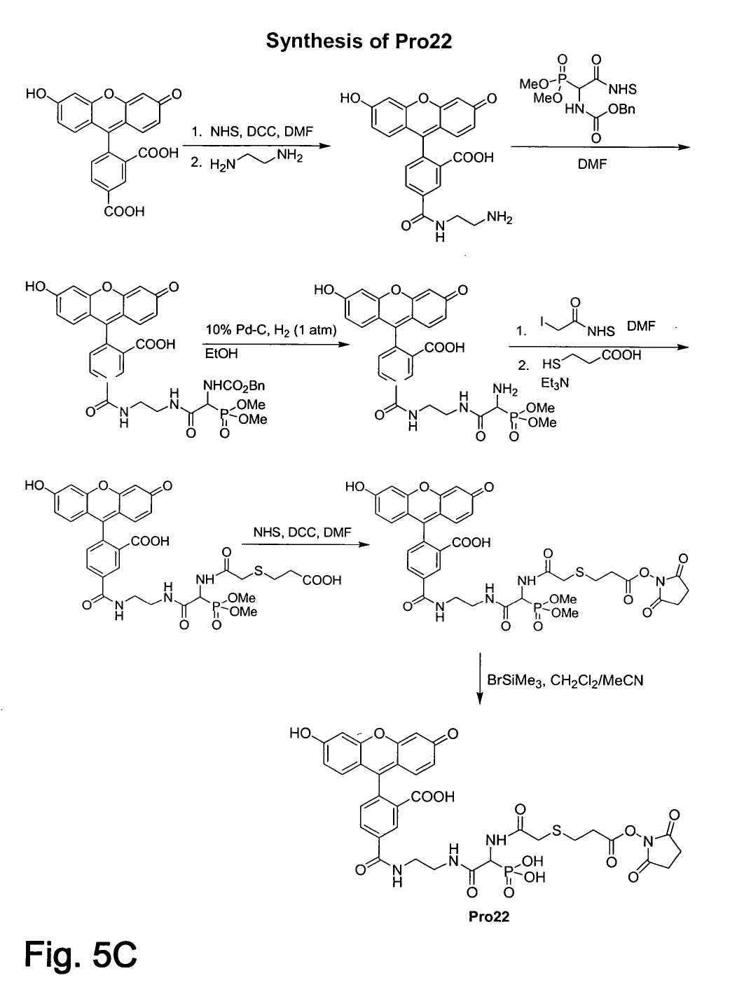

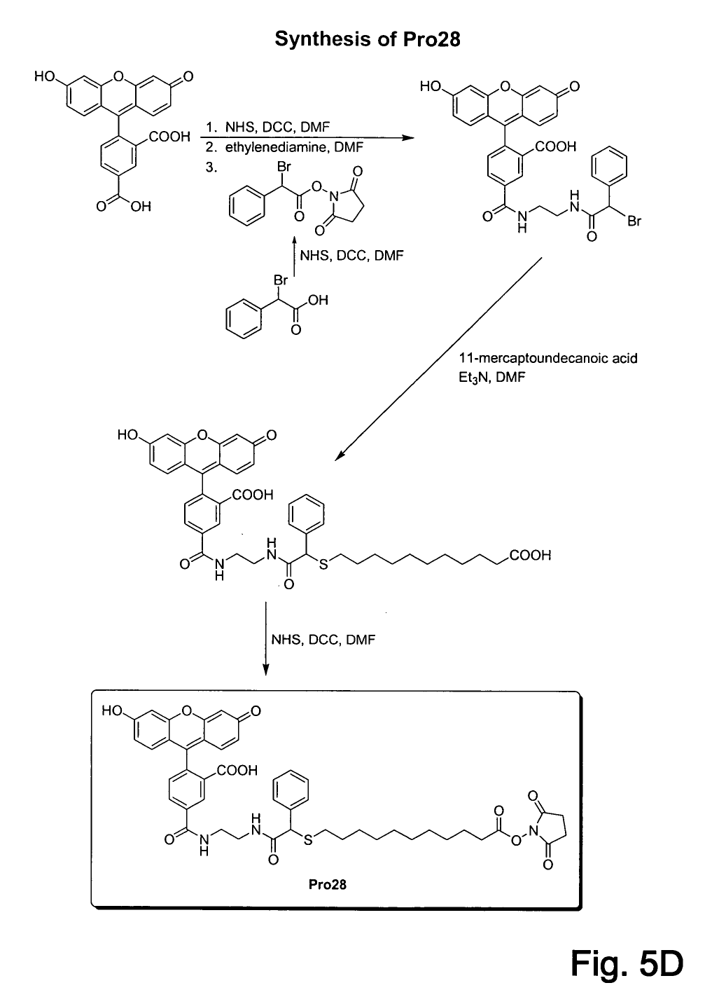

Binding compounds for use of the invention are synthesized as disclosed in the following references, which are incorporated herein by reference: International patent publications WO 00/66607; WO 01/83502; WO 02/95356; WO 03/06947; and U.S. Pat. Nos. 6,322,980; 6,514,700; and 6,627,400. Exemplary reagents for synthesis of binding compounds are shown in FIGS. 4 A-J. Exemplary synthesis protocols are illustrated in FIGS. 5A-5D.

### Cleavage-Inducing Moieties Producing Active Species

A cleavage-inducing moiety, or cleaving agent, is a group that produces an active species that is capable of cleaving a cleavable linkage, preferably by oxidation. Preferably, the active species is a chemical species that exhibits short-lived activity so that its cleavage-inducing effects are only in the proximity of the site of its generation. Either the active species is inherently short lived, so that it will not create significant background because beyond the proximity of its creation, or a scavenger is employed that efficiently scavenges the active species, so that it is not available to react with cleavable linkages beyond a short distance from the site of its generation. Illustrative active species include singlet oxygen, hydrogen peroxide, NADH, and hydroxyl radicals, phenoxy radical, superoxide, and the like. Illustrative quenchers for active species that cause oxidation include polyenes, carotenoids, vitamin E, vitamin C, amino acid-pyrrole N-conjugates of tyrosine, histidine, and glutathione, and the like, e.g. Beutner et al, Meth. Enzymol., 319: 226-241 (2000).

An important consideration for the cleavage-inducing moiety and the cleavable linkage is that they not be so far removed from one another when bound to a target protein that the active species generated by the sensitizer diffuses and loses its activity before it can interact with the cleavable linkage. Accordingly, a cleavable linkage preferably are within 1000 nm, preferably 20-200 nm of a bound cleavage-inducing moiety. This effective range of a cleavage-inducing moiety is referred to herein as its “effective proximity.”

Generators of active species include enzymes, such as oxidases, such as glucose oxidase, xanthene oxidase, D-amino acid oxidase, NADH-FMN oxidoreductase, galactose oxidase, glyceryl phosphate oxidase, sarcosine oxidase, choline oxidase and alcohol oxidase, that produce hydrogen peroxide, horse radish peroxidase, that produces hydroxyl radical, various dehydrogenases that produce NADH or NADPH, urease that produces ammonia to create a high local pH.

A sensitizer is a compound that can be induced to generate a reactive intermediate, or species, usually singlet oxygen. Preferably, a sensitizer used in accordance with the invention is a photosensitizer. Other sensitizers included within the scope of the invention are compounds that on excitation by heat, light, ionizing radiation, or chemical activation will release a molecule of singlet oxygen. The best known members of this class of compounds include the endoperoxides such as 1,4-biscarboxyethyl-1,4-naphthalene endoperoxide, 9,10-diphenylanthracene-9,10-endoperoxide and 5,6,11,12-tetraphenyl naphthalene 5,12-endoperoxide. Heating or direct absorption of light by these compounds releases singlet oxygen. Further sensitizers are disclosed in the following references: Di Mascio et al, FEBS Lett., 355: 287 (1994)(peroxidases and oxygenases); Kanofsky, J.Biol. Chem. 258: 5991-5993 (1983)(lactoperoxidase); Pierlot et al, Meth. Enzymol., 319: 3-20 (2000)(thermal lysis of endoperoxides); and the like.

Attachment of a binding agent to the cleavage-inducing moiety may be direct or indirect, covalent or non-covalent and can be accomplished by well-known techniques, commonly available in the literature. See, for example, “Immobilized Enzymes,” Ichiro Chibata, Halsted Press, New York (1978); Cuatrecasas, J. Biol. Chem., 245:3059 (1970). A wide variety of functional groups are available or can be incorporated. Functional groups include carboxylic acids, aldehydes, amino groups, cyano groups, ethylene groups, hydroxyl groups, mercapto groups, and the like. The manner of linking a wide variety of compounds is well known and is amply illustrated in the literature (see above). The length of a linking group to a binding agent may vary widely, depending upon the nature of the compound being linked, the effect of the distance on the specific binding properties and the like.

The cleavage-inducing moiety may be associated with the support by being covalently or non-covalently attached to the surface of the support or incorporated into the body of the support. Linking to the surface may be accomplished as discussed above. The cleavage-inducing moiety may be incorporated into the body of the support either during or after the preparation of the support. In general, the cleavage-inducing moiety is associated with the support in an amount necessary to achieve the necessary amount of active species. Generally, the amount of cleavage-inducing moiety is determined empirically.

As mentioned above, the preferred cleavage-inducing moiety in accordance with the present invention is a photosensitizer that produces singlet oxygen. As used herein, “photosensitizer” refers to a light-adsorbing molecule that when activated by light converts molecular oxygen into singlet oxygen. Photosensitizers may be attached directly or indirectly, via covalent or non-covalent linkages, to the binding agent of a class-specific reagent. Guidance for constructing of such compositions, particularly for antibodies as binding agents, available in the literature, e.g. in the fields of photodynamic therapy, immunodiagnostics, and the like. The following are exemplary references: Ullman, et al., Proc. Natl. Acad. Sci. USA 91, 5426-5430 (1994); Strong et al, Ann. New York Acad. Sci., 745: 297-320 (1994); Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; and the like.

Likewise, there is guidance in the literature regarding the properties and selection of photosensitizers suitable for use in the present invention. The following are exemplary references: Wasserman and R. W. Murray. Singlet Oxygen. (Academic Press, New York, 1979); Baumstark, Singlet Oxygen, Vol. 2 (CRC Press Inc., Boca Raton, Fla. 1983); and Turro, Modern Molecular Photochemistry (University Science Books, 1991).

The photosensitizers are sensitizers for generation of singlet oxygen by excitation with light. The photosensitizers include dyes and aromatic compounds, and are usually compounds comprised of covalently bonded atoms, usually with multiple conjugated double or triple bonds. The compounds typically absorb light in the wavelength range of about 200 to about 1,100 nm, usually, about 300 to about 1,000 nm, preferably, about 450 to about 950 nm, with an extinction coefficient at its absorbance maximum greater than about 500 M⁻¹ cm⁻¹, preferably, about 5,000 M⁻¹ cm⁻¹, more preferably, about 50,000 M⁻¹ cm⁻¹, at the excitation wavelength. The lifetime of an excited state produced following absorption of light in the absence of oxygen will usually be at least about 100 nanoseconds, preferably, at least about 1 millisecond. In general, the lifetime must be sufficiently long to permit cleavage of a linkage in a reagent in accordance with the present invention. Such a reagent is normally present at concentrations as discussed below. The photosensitizer excited state usually has a different spin quantum number (S) than its ground state and is usually a triplet (S=1) when the ground state, as is usually the case, is a singlet (S=0). Preferably, the photosensitizer has a high intersystem crossing yield. That is, photoexcitation of a photosensitizer usually produces a triplet state with an efficiency of at least about 10%, desirably at least about 40%, preferably greater than about 80%.

Photosensitizers chosen are relatively photostable and, preferably, do not react efficiently with singlet oxygen. Several structural features are present in most useful photosensitizers. Most photosensitizers have at least one and frequently three or more conjugated double or triple bonds held in a rigid, frequently aromatic structure. They will frequently contain at least one group that accelerates intersystem crossing such as a carbonyl or imine group or a heavy atom selected from rows 3-6 of the periodic table, especially iodine or bromine, or they may have extended aromatic structures.

A large variety of light sources are available to photo-activate photosensitizers to generate singlet oxygen. Both polychromatic and monchromatic sources may be used as long as the source is sufficiently intense to produce enough singlet oxygen in a practical time duration. The length of the irradiation is dependent on the nature of the photosensitizer, the nature of the cleavable linkage, the power of the source of irradiation, and its distance from the sample, and so forth. In general, the period for irradiation may be less than about a microsecond to as long as about 10 minutes, usually in the range of about one millisecond to about 60 seconds. The intensity and length of irradiation should be sufficient to excite at least about 0.1% of the photosensitizer molecules, usually at least about 30% of the photosensitizer molecules and preferably, substantially all of the photosensitizer molecules. Exemplary light sources include, by way of illustration and not limitation, lasers such as, e.g., helium-neon lasers, argon lasers, YAG lasers, He/Cd lasers, and ruby lasers; photodiodes; mercury, sodium and xenon vapor lamps; incandescent lamps such as, e.g., tungsten and tungsten/halogen; flashlamps; and the like. By way of example, a photoactivation device disclosed in Bjornson et al, International patent publication WO 03/051669 is employed. Briefly, the photoactivation device is an array of light emitting diodes (LEDs) mounted in housing that permits the simultaneous illumination of all the wells in a 96-well plate. A suitable LED for use in the present invention is a high power GaAIAs IR emitter, such as model OD-880W manufactured by OPTO DIODE CORP. (Newbury Park, Calif.).

Examples of photosensitizers that may be utilized in the present invention are those that have the above properties and are enumerated in the following references: Singh and Ullman, U.S. Pat. No. 5,536,834; Li et al, U.S. Pat. No. 5,763,602; Martin et al, Methods Enzymol., 186: 635-645 (1990);Yarmush et al, Crit. Rev. Therapeutic Drug Carrier Syst., 10: 197-252 (1993); Pease et al, U.S. Pat. No. 5,709,994; Ullman et al, U.S. Pat. No. 5,340,716; Ullman et al, U.S. Pat. No. 6,251,581; McCapra, U.S. Pat. No. 5,516,636; Thetford, European patent publ. 0484027; Sessler et al, SPIE, 1426: 318-329 (1991); Magda et al, U.S. Pat. No. 5,565,552; Roelant, U.S. Pat. No. 6,001,673; and the like.

As with sensitizers, in certain embodiments, a photosensitizer may be associated with a solid phase support by being covalently or non-covalently attached to the surface of the support or incorporated into the body of the support. In general, the photosensitizer is associated with the support in an amount necessary to achieve the necessary amount of singlet oxygen. Generally, the amount of photosensitizer is determined empirically.

### Membrane-Bound Photosensitizers

Lipophilic sensitizers can be incorporated into lipid membranes in an orientation and manner similar to that of phospholipids where the hydrophobic moiety comprising of the hydrocarbon chains can orient inward and the more hydrophilic entities can orient outwards. Thus, as in the usual cellular membranes, the hydrocarbon portion of the lipophilic sensitizer can be incorporated into the lipid environment whereas the hydrophilic sensitizer portion can be exposed to the aqueous interface at the membrane surface. Exemplary, lipophilic sensitizers are lipid-derivatized texaphyrins, e.g. U.S. Pat. No. 6,375,930.

Where intact cellular structures are required, the methods used to incorporate lipophilic sensitizers into the cells preferably cause minimal disruption of the cell and of the integrity of membranes. In addition, the cells can be fixed and treated with routine histochemical or cytochemical procedures, where the procedure preferably does not affect the incorporation.

The membranes can be labeled with lipophilic sensitizers according to the method described in Barak and Webb (1981) J. Cell Biol. 90:595-604. Typically, the membrane, such as the intact cell, is contacted with the compounds of the invention, preferably in an aqueous media. The aqueous media can be water, water and organic solvent, such as DMSO, DMF, DMA, or a mixture thereof, and can contain buffers such as phosphate, acetate, tris, and the like. The membranes and lipophilic sensitizer are contacted for between 1 min. to about 1 week, preferably about 1 h to 76 h, more preferably about 2 h to about 48 h, or any integer in between. The formulations may additionally be subjected to chemical or mechanical treatment, such as the addition of a surfactant (Tween 80, for example), shaking, stirring, electroporation, and the like. Alternatively, the formulation can be heated to about 30° C. to 50° C., preferably about 35° C. to about 40° C., until labeling is achieved. After labeling, the unbound components can be removed by washing, or by centrifugation, for example, and the sensitizer-labeled cells or membranes isolated.

Alternatively, a capture moiety, such as biotin, having a lipophilic group may be anchored in a membrane followed by conjugation to avidin or streptavidin and finally by attachment of a biotinylated sensitizer via the avidin or streptavidin, as illustrated in FIG. 1G. Cells (1301) are combined with biotin having a lipophilic moiety (1304) (referred to below as biotin-G) to form a population of cells (1306) having membranes containing free biotin. To this population is added avidin or streptavidin (1310) to form biotin-avidin or biotin-streptavidin complexes (1312) on the cell surfaces. These cells are then combined (1313) with biotinylated photosensitizers to form complexes (1312) on the cell surfaces that comprise sensitizer-treated membranes.

### Cell Types

The cell(s) used in the methods described herein can be of any origin, including from prokaryotes, eukaryotes, or archeons, but preferably contain membranes that are lipophilic. The cell(s) may be living or dead. If obtained from a multicellular organism, the cell may be of any cell type. Thus, the cell(s) may be a cultured cell line or a primary isolate, the cell(s) may be mammalian, amphibian, reptilian, plant, yeast, bacterium, spirochetes, or protozoan. The cell(s) may be, for example, human, murine, rat, hamster, chicken, quail, goat or dog. The cell may be a normal cell, a mutated cell, a genetically manipulated cell, a tumor cell, hybridomas that are positive for secretion of selected antibodies, and the like. Of particular interest are membranes obtained from the type of cell that differentially expresses (over-expresses or under-expresses) a disease-causing gene. As is apparent to one skilled in the art, various cell lines, such as CHO, for example, may be obtained from public or private repositories. T he largest depository agent is American Type Culture Collection (http://www.atcc.org), which offers a diverse collection of well-characterized cell lines derived from a vast number of organisms and tissue samples. Preferably, cells analyzed by the method and system of the invention are mammalian cells, and more preferably, human cells. Human cells may be normal or may be from diseased tissue, such as a neoplasm or tumor.

Exemplary cell types from multicellular organisms include acidophils, acinar cells, pinealocytes, adipocytes, ameloblasts, astrocytes, basal (stem) cells, basophils, hepatocytes, neurons, bulging surface cells, C cells, cardiac muscle cells, centroacinar cells, chief cells; chondrocytes, Clara cells, columnar epithelial cells, corpus luteal cells, decidual cells, dendrites, endrocrine cells, endothelial cells, enteroendocrine cells, eosinophils, erythrocytes, extraglomerular mesangial cells, fetal fibroblasts, fetal red blood cells, fibroblasts, follicular cells, ganglion cells, giant Betz cells, goblet cells, hair cells, inner hair cells, type I hair cells, hepatocytes, endothelial cells, Leydig cells, lipocytes, liver parenchymal cells, lymphocytes, lysozyme-secreting cells, macrophages, mast cells, megakaryocytes, melanocytes, mesangial cells, monocytes, myoepithelial cells, myoid cells, neck mucous cells, nerve cells, neutrophils, oligodendrocytes, oocytes, osteoblasts, osteochondroclasts, osteoclasts, osteocytes, pillar cells, sulcal cells, parathyroid cells, parietal cells, pepsinogen-secreting cells, pericytes, pinealocytes, pituicytes, plasma cells, platelets, podocytes, spermatocytes, Purkinje cells, pyramidal cells, red blood cells, reticulocytes, Schwann cells, Sertoli cells, columnar cells, skeletal muscle cells, smooth muscle cells, somatostatin cells, enteroendocrine cells, spermatids, spermatogonias, spermatozoas, stellate cells, supporting Deiter cells, support Hansen cells, surface cells, surface epithelial cells, surface mucous cells, sweat gland cells, T lymphocytes, theca lutein cells, thymocytes, thymus epithelial cell, thyroid cells, transitional epithelial cells, type I pneumonocytes, and type II pneumonocytes.

### Microfluidics System

The fabrication and operation of microfluidics systems for moving and manipulating fixed or living cells is well-known in the art, as exemplified by the following references: Swanek et al, chapter 17, and Lillard et al, chapter 18, Landers, editor, Handbook of Capillary Electrophoresis, Second Edition (CRC Press, Boca Raton, 1996); McClain et al, Anal. Chem., 75: 5646-5655 (2003); Dittrich et al, Anal. Chem., 75: 5767-5774 (2003); Li and Harrison, Anal. Chem., 69: 1564-1568 (1997); Chiem and Harrison, Clinical Chem., 44: 591-598 (1998); Ramsey, U.S. Pat. No. 5,858,195; Spence et al, U.S. Pat. No. 6,540,895; Nelson et al, U.S. Pat. No. 6,613,525; and the like.

A sample of cells whose membane-associated analytes are to be measured is introduced into a reservoir of a microfluidic device. The reservoir is connected to a channel or other reservoirs, which channel may have branches and connections to other channels, where the channels will be subject to electrokinetic forces for movement of the cells in the channels and for contacting the cells with agents for performance of an operation. The cells can be moved in small discrete groups or individually from the reservoir into a channel and processed in accordance with the operation and the result of the operation then determined. A detector is provided which views the medium at a site in the channel to determine the status of the cells as a result of the interaction with the agent(s). By “status” is intended any change in a characteristic of the cell, which can be detected, including the binding of agents, changes in physiologic pathways, viability of the cells, mitotic status, shape, etc.

Cells may be moved within a microfluidic device by various means, including pneumatically or pressure driven and electrokinetically. Using electrokinesis, there are three ways to move cells in capillary channels: electrophoresis, electroosmotic force and electroosmotic pumping. In the case of electrophoresis, the cells will be subject to an electrical field and desirably a low salt concentration buffer will be used to prevent overheating that would occur in a high salt medium, which is the medium desired for viable cells. In this situation, one will normally use fixed cells, which will retain their receptor binding characteristics in the low salt medium and may be transported in the channel based on their mass/charge ratio. For moving the cells by electroosmotic force, again the cells will be subject to an electrical field, but the movement of the cells will be driven by the electroosmotic force, so the cells will move both as a function of their charge and the electroosmotic flow which is independent of the cellular charge. The electroosmotic pump is connected to the cell channel, where the cell channel is not in an electrical field. By having fluid flow in the pump in one or two legs, where the flow is directed away from the cell-containing channel, the cells can move toward the electroosmotic pump along with any reagents in a medium appropriate for cell viability.

The cells may be subjected to different buffers, depending upon the nature of the cells, the operation, whether the cells are viable or fixed, the exposure time for the cells, and the like. For viable cells, one will normally employ a relatively high salt concentration as compared to the most advantageous salt concentration for electrokinesis. Therefore, when using viable cells, desirably one will use, when appropriate, phosphate buffered saline, HEPES, MOPS, Tris, etc., pH 7.0 to 7.5, where the salt concentration, e.g. NaCl, KCl, etc., will generally be at least about 100 mM and not more than about 200 mM. In addition, from about 5-20% fetal calf serum or other proteinaceous composition may be present. By contrast, the salt concentration for electrokinesis will generally be below about 100 mM, since at higher conductivities the voltage has to be relatively low to prevent overheating the capillary and consequently the movement of charged species is slower. For the electrokinesis, one would prefer to have a low conductivity and work at about 10 to 50 mM salt.

The parameters to be considered are the desired speed of the operation, the viability of the cells, the susceptibility of the cells to lower salt concentrations, the effect of the addition of agents to increase osmotic pressure of the buffer medium, whether electrophoresis or electroosmotic force (“EOF”) is being used, where the latter may be distant from the area of the cellular operation, the duration of the operation where the cells are exposed to the nonphysiologic osmolality, and the like. To maintain osmolality or an isotonic medium, one may use neutral compounds, including zwitterions, which are physiologically acceptable. Such compounds include saccharides, e.g. glucose, dextran, cyclodextrin, etc., urea, polyalkylene oxides, polyols, betaines, cholines, silanols, etc. The concentration of the osmolality enhancing agents will vary with the agent to provide a physiologic osmolality in the range of about 250 to 350 mOsm.

For the use of electrophoresis for movement of the cells, the medium will usually be non-physiologic, that is the buffer salt concentration will be marginal or less than is normally used in growth media for cells. Electrophoresis will preferably be used for fixed cells. Most cells are negative, so that the downstream electrode will be positive. Depending on the nature of the agents, they may be combined with the cells in the reservoir, introduced into a channel from a branched channel, where the branch is positioned to encounter the cells at a cross-section, to overtake the cells, as they migrate down a channel, or encounter the cells by moving in the opposite direction of the cells in the channel. Alternatively, one may provide for the agent(s) to be in the medium, so that there will be a substantially uniform concentration of the agent(s) throughout the channel network.

In one application of electroosmotic force for movement of the cells, an electroosmotic pump is employed to move the cells from one site to another. By using the electroosmotic pump, the salt medium for the viable cells may be maintained in the range of 100 to 200 mM, while the salt medium in the electroosmotic pump will be in the range of about 10 to 50 mM. The pump may be created by having at least three legs (channels), where two legs with oppositely charged walls are connected at the same site to the channel in which the cells are to be moved. Alternatively, the walls may be may be neutral or slightly charged and a charged polymer employed which provides for dynamic coating of the walls.

The connection of the channels may be a T-shape, a Y-shape, or X-shape, where any one of the arms may be selected as the channel in which the cells are moved. In addition, there may be a channel for the cells and one or more channels for reagents to mix with the cells in the main channel. All of these channels would be subject to the EOP and be pulled together into a main channel which would move the cells to the detector.

The walls of the channels of the EOP may be charged as a result of the composition of the walls, where neutral walls may be modified to obtain the desired charge or the substantially permanent coating of the walls with polymeric compositions of a particular charge or by “dynamic” coating of the walls. For a description of an EOF pump using different forms of charged walls, see WO 98/46438 and U.S. Pat. no. 5,858,195, which are specifically incorporated herein by reference. Methods taught for providing charged surfaces are the use of detergents with a partially solubilized polymeric wall, modifying or derivatizing the polymeric surface, either chemically or by plasma irradiation, etc., coatings, and the like. The surface should have a zeta potential to provide an electroosmotic mobility (μEO) of at least about 2×10⁻⁵ cm²V⁻¹s⁻¹, more usually at least about 5×10⁻⁵ cm²V⁻¹s⁻¹. (μEO=v/E, where v is the velocity of the fluid and E is the electric field strength; Q=vA, where Q is the volume velocity of the fluid and A is the cross-sectional area of the channel through which the fluid is flowing.) By having two legs in the EOP, each leg pumping fluid from the cell channel, one can more rapidly move the cells down the cell channel. The channels associated with the pump will have a cross-section, which is capable of accommodating the flow of liquid in the different channels associated with the cells and the reagents.

The electrokinesis buffer for use with the hydrophilic charged polymer may be a conventional buffer such as, for example, the Good's buffers (HEPES, MOPS, MES, Tricine, etc.), and other organic buffers (Tris, acetate, citrate, and formate), including standard inorganic compounds (phosphate, borate, etc.). Exemplary buffer systems include: (i) 100 mM sodium phosphate, pH 7.2; (ii) 20 mM tris-base, 20 mM boric acid, 2 mM ETDA, pH 8.3; or (iii) 25 mM Hepes, pH 7.5. The buffer may also contain any other material that does not interfere with the functional behavior of the buffer, the charged hydrophilic polymer, the microfluidic processing to be conducted, and so forth.

For charged walls, the surface that contacts the electrokinesis buffer, such as, e.g., inner surfaces of channels within a microfluidic device, may be activated with base before use with the electrokinesis buffer of the invention. In the presence of a basic medium, some plastic surfaces generate carboxyl groups on the surface. Glass and fused silica surfaces have silanol groups on the surface. Both carboxyl groups and silanol groups are negatively charged and this negative charge aids in ionic interaction between these groups and the positively charged hydrophilic polymers of the invention. Such ionic interaction contributes to the superior surface coverage of the hydrophilic polymers, which bind transiently to, and prevent adsorption of proteins and hydrophobic compounds to, the channel surfaces. Basic media that may be employed to achieve such activation include, for example, aqueous sodium hydroxide, potassium hydroxide, and the like. The concentration of the basic medium should be sufficient to achieve the level of activation or hydrolysis desired. Usually, the level of hydrolysis will provide an EOF under the conditions described in the Experimental section in the range of about −3 to −50×10⁻⁵ cm²/V sec. In this regard and from another viewpoint, the level of hydrolysis should be sufficient to achieve the desired EOF.

The concentration of cells in the sample reservoir will usually be at least about 10³ per ml, usually in the range of about 10⁴ to 10⁸ per ml. One will usually have at least about 10² cells, more usually at least about 10³ cells and will usually have fewer than about 10⁶ cells.

One may use viable cells, fixed cells, natural cells, cell lines, genetically modified cells, hybridomas, or the like. Genetically modified cells may provide for predetermined, including enhanced, levels of a cell surface receptor, formation of a detectable product upon effective binding of a ligand to the cell surface receptor, novel physiological pathways, immortalization, or the like. Any of the different kinds of cells may be used where binding is the endpoint, although fixed cells will be the most convenient. Cells may be readily fixed in accordance with conventional ways, using formaldehyde or glutaraldehyde or other fixatives. Genetically modified cells may be modified by providing for expression of a fusion protein, where the protein is fused to a fluorescent protein, such as green fluorescent protein, or an enzyme, which has a product providing a detectable signal, such as β-galactosidase or luciferase. See, for example, U.S. Pat. Nos. 5,436,128 and 5,897,990. Alternatively, cells may be loaded with ionic responsive dyes, such as CA responsive dyes. See, for example, U.S. Pat. No. 5,049,673.

The cells in the reservoir may be combined with various agents for incubation or reaction. Prior to, concomitant with, or subsequent to introduction into the reservoir, the cells may be exposed to one or more agents for the performance of the operation. Incubations may be carried out prior to introduction of the cells into the reservoir or while the cells are in the reservoir. Agents may be added to the reservoir to allow for equilibrium or for kinetic determinations, as appropriate. A common use of cells is to determine activity of an agent, e.g. a ligand, where the determination may be as a result of a physiological effect of the agent or a competition between the agent and a competitive labeled reagent. For example, for test compound screening, one may add to the cells, the test compound and a known ligand for a cellular receptor, concurrently or consecutively, where there may be displacement or competition for available binding sites. The cells may then be removed from the reservoir into a channel. By having a side channel, which substantially dilutes the buffer to provide a conductivity of the medium appropriate to electrokinesis, further reaction may be quenched and the extent of the reaction determined. By using a labeled ligand, and determining the level of binding of the ligand to the cell, one can evaluate the binding affinity of the test compound. Where one is interested in transduction by the binding of the test compound to a receptor, with activation of messengers and/or transcription factors, cells which provide for a detectable signal naturally or as a result of genetic modification may be employed. Where one is interested in the level of binding, the off-rate of the ligand bound to the receptor is usually slow, so that the cells may be moved to a site for detection and the time differential between reading individual cells will not significantly change the observed result.

Instead of adding the agent(s) to the cells before the cells enter a channel, the agent(s) may be added to the cells in the channel. In this way, all cells will be exposed to the agent(s) for about the same time prior to making the determination. By having one or more side channels, which comprise sources of one or more agents, different stages of the operation may be performed as the cells move through a channel or channel network. The agents may be injected into the channel as small plugs, e.g. 10 to 500 nl, where the plug will mix with the medium carrying the cells.

In carrying out the operation, EOF pumping may be employed for moving the cells from the cell reservoir into a channel and through a channel network. The cells need not be exposed to non-physiologic medium and the pump may be downstream from the cell reservoir. By pumping fluid with the EOF pump, the cells will be drawn into a channel, where agents for the operation may also be pumped into a common channel, where the agents will act on the cells. The agents may be pumped by positive pumping, so that each agent would have its own metering or the agents may be drawn into the channel by a common pump downstream from the agent source, where the mixing of the cells and agent(s) would be based on the relative sizes of the channels. By using concentrated solutions of agents, one can provide for positive pumping using low conductivity solutions, without significantly disturbing the conductivity of the medium in the channel supporting the cells.

The microfluidic device which is employed will have a plurality of reservoirs, at least two, and at least one channel. The device may have a plurality of microfluidic units, particularly where the device interfaces with a microtiter well plate. The reservoirs will serve as cell reservoirs, agent reservoirs, waste reservoirs, etc. The reservoirs will generally have volume capacities of from about 0.1 to 20 μl, more usually from about 1 to 10 μl, to accommodate the desired number of cells, the volume of agents, and waste. The reservoirs will generally have a depth of about 0.2 to 5 mm. The channels will vary in depth and width, generally having cross-sections in the range of about 100 to 10,000, more usually about 200 to 5,000μ², where the depth will generally be in the range of about 10 to 100μ, and the width in the range of about 10 to 250μ, where the width will generally be greater than the depth. The materials employed for the device include silicon, glass and plastics, particularly acrylates, although other materials may also be involved, as supports, heat transfer, and the like. Generally, the device will comprise a substrate and a cover, where the substrate will generally have a thickness in the range of about 25μ to 5 mm and an adhering cover to enclose the channels, which will generally be of a thickness in the range of about 10 to 100μ. Desirably, the surface of the substrate will be planar. Various openings may be provided in the cover or the substrate, to provide access to the reservoirs and provide access for the electrodes. The manner of use of the device is conventional.

Voltage differentials which are employed for the transport of the cells will generally be in the range of about 100 to 2000V, more usually about 100 to 1000V. Cells may be moved at speeds varying in the range of about 10 to 10⁴ cells/min past a detection point.

Often, a microfluidic network is employed, which is a system of interconnected cavity structures and capillary-size channels configured with a plurality of branches through which fluids may be manipulated and processed. A cavity structure is an unfilled space, preferably, a hollowed out space in an object, such as, e.g., a planar substrate, a plate, or the like such as, for example, a well, a reservoir, an incubation chamber, a separation chamber, an enrichment chamber, a detection chamber, and the like. The cavity structures are usually present at one or both of the termini, i.e., either end, of a channel. The cavity structures may serve a variety of purposes, such as, for example, means for introducing a buffer solution, elution solvent, reagent rinse and wash solutions, and so forth into a main channel or one or more interconnected auxiliary channels, receiving waste fluid from the main channel, and the like. Channels provide a conduit or means of communication, usually fluid communication, more particularly, liquid communication, between elements of the present apparatus. The elements in communication are, e.g., cavity structures, and the like. Channels include capillaries, grooves, trenches, microflumes, and so forth. The channels may be straight, curved, serpentine, labyrinth-like or other convenient configuration within the planar substrate. The cross-sectional shape of the channel may be circular, ellipsoid, square, rectangular, triangular and the like so that it forms a microchannel within the substrate in which it is present.

The channels and the like are usually of capillary dimension, i.e., having a cross-sectional area that provides for capillary flow through a channel. At least one of the cross-sectional dimensions, e.g., width, height, diameter, is at least about 1 μm, usually at least 10 μm, and is usually no more than 500 μm, preferably no more than 200 μm. Channels of capillary dimension typically have an inside bore diameter (ID) of from about 1 to 200 microns, more typically from about 25 to 100 microns, with cross-sections of greater than 100 microns, generally in the range of about 250 to 5000 microns.

### Separation of Released Molecular Tags

Preferably, molecular tags are electrophoretically separated to form an electropherogram in which the separated molecular tags are represented by distinct peaks.

Methods for electrophoresis of are well known and there is abundant guidance for one of ordinary skill in the art to make design choices for forming and separating particular pluralities of molecular tags. Guidance for capillary electrophoretic systems and analysis is found in Landers, editor, Handbook of Capillary Electrophoresis, Second Edition (CRC Press, Boca Raton, 1997); and in U.S. Pat. Nos. 5,374,527; 5,624,800; 5,552,028; and the like.

In one aspect of the invention, during or after electrophoretic separation, the molecular tags are detected or identified by recording fluorescence signals and migration times (or migration distances) of the separated compounds, or by constructing a chart of relative fluorescent and order of migration of the molecular tags (e.g., as an electropherogram). Preferably, the presence, absence, and/or amounts of molecular tags are measured by using one or more standards as disclosed by Williams et al, U.S. patent publication Ser. No. 2003/0,170,734A1, which is incorporated herein by reference. During or after separation, fluorescent molecular tags can be illuminated by standard means, e.g. a high intensity mercury vapor lamp, a laser, or the like. Typically, the molecular tags are illuminated by laser light generated by a He—Ne gas laser or a solid-state diode laser. The fluorescence signals can then be detected by a light-sensitive detector, e.g., a photomultiplier tube, a charged-coupled device, or the like. Exemplary electrophoresis detection systems are described elsewhere, e.g., U.S. Pat. Nos. 5,543,026; 5,274,240; 4,879,012; 5,091,652; 6,142,162; or the like. In another aspect, molecular tags may be detected electrochemically detected, e.g. as described in U.S. Pat. No. 6,045,676.

What is claimed is:

1. A method for determining the population of each of a plurality of membrane-associated analytes of a cell, the method comprising the steps of:
providing a column of buffer solution moving along an electrophoresis channel, the channel having in succession a cleavage zone and a separation zone and having an electrical field collinear with the moving column of buffer solution;
inserting a cell into the moving column of buffer solution upstream of the cleavage zone so that the cell is transported by the moving column of buffer solution through the electrophoresis channel to the cleavage zone,
wherein the cell comprises, attached to each of the plurality of membrane-associated analytes, a binding composition specific for that membrane-associated analyte and having one or more molecular tags, each attached to the binding composition by a cleavable linkage, wherein the molecular tags of different binding compositions have different electrophoretic mobilities, so that distinct peaks are formed upon electrophoretic separation of a mixture of the tags;
cleaving the cleavable linkages in the cleavage zone to release the molecular tags from the cell; and
electrophoretically separating and identifying the released molecular tags in the moving column of buffer solution in the separation zone of the electrophoresis channel, to determine the populations of membrane-associated analytes of the cell.

2. The method of claim 1, wherein a sequence of cells is inserted into the moving column of buffer solution upstream of the cleavage zone, at a frequency such that only one cell is present in the cleavage zone at any given time.

3. The method of claim 2, where electrophoretically separating the released molecular tags produces an electropherogram having a series of discrete groups of signals from said released molecular tags, each group corresponding to a single cell.

4. The method of claim 1, wherein said cell is inserted into the moving column of buffer solution via a side channel which intersects the electrophoresis channel upstream of the cleavage zone, said side channel being in fluid communication with a sample reservoir.

5. The method of claim 4, wherein said electrophoresis channel is in fluid communication with a buffer reservoir, located upstream of said side channel.

6. The method of claim 6, wherein pressure is applied simultaneously to said sample reservoir and said buffer reservoir.

7. The method of claim 1, wherein said cleavable linkage is photochemically cleavable.

8. The method of claim 1, wherein said cell comprising said binding compounds is prepared by contacting a cell with a plurality of binding compounds, each having a binding moiety specific for a different membrane-associated analyte, wherein said cell includes at least one of said membrane-associated analytes.

9. The method according to any of claims 1 through 8 wherein said cell is a human cell.

10. The method of claim 9 wherein at least one of said membrane-associated analytes is a cell membrane receptor.

11. The method of claim 10 wherein said cell membrane receptor is an ErbB receptor.

12. The method of claim 9 wherein at least one of said membrane-associated analytes is a receptor dimer.

13. The method of claim 12 wherein said receptor dimer is a Her dimer.

14. A microfluidics system for determining the population of each of a plurality of membrane-associated analytes of a cell, comprising:
an electrophoresis channel containing a moving column of buffer solution, the electrophoresis channel having in succession a cleavage zone, a separation zone, and a detection zone, and having an electrical field collinear with the moving column of buffer solution;
a sample-injection channel for inserting a cell into the moving column of buffer solution upstream of the cleavage zone so that the cell is transported by the moving column of buffer solution through the electrophoresis channel to the cleavage zone;
wherein the cell comprises, attached to each of the plurality of membrane-associated analytes, a binding compound specific for the membrane-associated analyte and having one or more molecular tags, each attached to the binding compound by a cleavable linkage, wherein the molecular tags of different binding compounds have different electrophoretic mobilities, so that distinct peaks are formed upon electrophoretic separation of a mixture of molecular tags;
a light source having a light beam directed to the cleavage zone for directly or indirectly cleaving the cleavable linkages in the cleavage zone to release the molecular tags from the cell; and
a detector for collecting a signal generated by electrophoretically separated molecular tags in the detection zone to determine the populations membrane-associated analytes of the cell.

15. The system of claim 14 wherein said cell is a human cell.

16. The method of claim 15 wherein at least one of said membrane-associated analytes is a cell membrane receptor.

17. The method of claim 16 wherein said cell membrane receptor is selected from the group consisting of receptor tyrosine kinases and G-protein coupled receptors.

18. The method of claim 17 wherein said cell membrane receptor is an ErbB receptor.

19. The method of claim 15 wherein at least one of said membrane-associated analytes is a receptor dimer.

20. The method of claim 12 wherein said receptor dimer is a Her dimer.
